(19) United States
(12) Patent Application Publication
(10) Pub. No.: US 2022/0304973 A1
(43) Pub. Date: Sep. 29, 2022

(54) METHODS FOR OPTIMIZED CANNABINOID DOSAGE DETERMINATION

(71) Applicant: Ojai Energetics PBC, Ojai, CA (US)

(72) Inventor: William KLEIDON, Ojai, CA (US)

(21) Appl. No.: 17/519,859

(22) Filed: Nov. 5, 2021

Related U.S. Application Data
(63) Continuation of application No. PCT/US20/31721, filed on May 6, 2020.
(60) Provisional application No. 62/844,371, filed on May 7, 2019.

(51) Int. Cl. A61K 31/352; G01N 33/50

(52) U.S. Cl. CPC A61K 31/352; G01N 33/5008; G01N 2800/60

(57) ABSTRACT

The present disclosure provides methods and kits for determining a target dosage of a cannabinoid composition. A kit for determining the target dosage may comprise the cannabinoid composition, an applicator device that dispenses said cannabinoid composition in a fixed volume, and instructions for determining the target dosage. The methods described may be unique to, or dependent on, a particular individual and a particular cannabinoid composition. Some methods described may comprise detecting changes in the taste or flavor of a cannabinoid composition to determine the target dosage of the cannabinoid composition.

### CROSS-REFERENCE

This application is a continuation of International Patent Application No. PCT/US20/31721, filed on May 6, 2020, claims the benefit of U.S. Provisional Patent Application No. 62/844,371, filed May 7, 2019, which application is entirely incorporated herein by reference.

### BACKGROUND

Cannabinoids are a class of chemicals that can bind with cannabinoid receptors. Cannabinoid receptor ligands include endocannabinoids, which can be found naturally occurring in humans and other animals, phytocannabinoids, which can be found in cannabis and other plants, and synthetic cannabinoids. Cannabinoids include tetrahydrocannabinol (THC), such as delta-9-tetrahydrocannabinol, and cannabidiol (CBD). At least 100 different cannabinoids have been isolated from cannabis plants.

Cannabinoids may be isolated from plants of the Cannabaceae family. Cannabinoids may have a range of pharmaceutical applications including pain relief, anti-emetic effects, and anti-inflammatory effects. Cannabinoids may be composed in a variety of forms, including oils, tinctures, and/or balms, depending upon the intended pharmaceutical application.

### SUMMARY

The present disclosure provides methods and kits for determining a target dosage of a given cannabinoid composition for a given individual. Variances between individuals and/or daily variations in biochemistry within an individual may affect the target dosage of a cannabinoid composition for a given individual at a given time. The method of the present disclosure may be a personalized method that permits each individual to determine their target dosage for a given cannabinoid composition. The target dosage amount may vary for a given individual on a day-to-day basis and the described methods may provide a method for frequently recalibrating the cannabinoid dosage to account for these variations. The provided methods may involve oral application of a cannabinoid composition, specifically in the sublingual region of the mouth. The target dosage may be correlated with certain biomarkers, such as age, weight, height, basal body temperature, resting heart rate, systolic blood pressure, diastolic blood pressure, blood sugar level, and oxidative stress level. Target dosage and biomarker data may be combined to determine the efficacy of cannabinoid health treatments or improve the accuracy of dosage determination.

An aspect of the present disclosure provides a kit comprising: a cannabinoid composition; an applicator device that dispenses the cannabinoid composition in a fixed volume; and instructions for determining a target dosage according to a method of: (a) measuring a first value of at least one biomarker for an individual; (b) using the applicator device to apply the cannabinoid composition in a first fixed volume to a region of a mouth of the individual; (c) determining a first taste for the cannabinoid composition, wherein the first taste is characterized by one or more members selected from the group consisting of salty, bitter, sweet, sour, and savory; (d) applying the cannabinoid composition in a second fixed volume to the region of the mouth of the individual; (e) determining a second taste for the cannabinoid composition, wherein the second taste is characterized by one or more members selected from the group consisting of salty, bitter, sweet, sour, and savory; and (f) upon detecting a change between characterization of the second taste and the first taste, (1) measuring a second value of the at least one biomarker for the individual, and (2) determining, if the second value meets a condition, that a cumulative volume of the cannabinoid composition applied to the region of the individual is indicative of the target dosage of the cannabinoid composition for the individual.

In some embodiments, the applicator device is configured to dispense the cannabinoid composition in an additional fixed volume, wherein the fixed volume and the additional fixed volume are different.

In some embodiments of any one of the kits provided herein, the kit further comprises a sensor device configured to measure a value of the at least one biomarker in a biological sample of an individual. In some embodiments, the sensor device is further configured to collect the biological sample from the individual. In some embodiments of any one of the kits provided herein, the biological sample comprises a blood sample.

In some embodiments of any one of the kits provided herein, the kit further comprises a sample collector device configured to collect a biological sample from an individual. In some embodiments, the biological sample comprises the at least one biomarker of the individual. In some embodiments, the biological sample comprises a blood sample.

In some embodiments of any one of the kits provided herein, the instructions for determining the target dosage comprises instructions for repeating (d) through (e) until the change is detected to determine the cumulative volume.

In some embodiments of any one of the kits provided herein, the cannabinoid composition comprises cannabidiol. In some embodiments of any one of the kits provided herein, the cannabinoid composition comprises tetrahydrocannabinol at less than 0.3% by weight.

In some embodiments of any one of the kits provided herein, the fixed volume is between about 1 milliliters (mL) to about 5 mL. In some embodiments of any one of the kits provided herein, the first fixed volume is between about 1 mL to about 5 mL. In some embodiments of any one of the kits provided herein, the first fixed volume and the second fixed volume are substantially the same. In some embodiments of any one of the kits provided herein, the second fixed volume is less than the first fixed volume.

In some embodiments of any one of the kits provided herein, the instructions for determining the target dosage comprises instructions for rinsing the mouth. In some embodiments, the instructions for determining the target dosage comprises instructions for rinsing prior to (b). In some embodiments, the instructions for determining the target dosage comprises instructions for rinsing prior to (d). In some embodiments, the instructions for determining the target dosage comprises instructions for rinsing subsequent to (b).

In some embodiments of any one of the kits provided herein, the region of the mouth comprises the sublingual region. In some embodiments of any one of the kits provided herein, the region of the mouth comprises the ventral region of the tongue.

In some embodiments of any one of the kits provided herein, the instructions comprise characterizing the first taste by at least bitter. In some embodiments of any one of the kits provided herein, the instructions comprise characterizing the second taste by at least sweet.

In some embodiments of any one of the kits provided herein, the instructions comprise determining a second dosage of the cannabinoid composition at a fixed interval. In some embodiments, the fixed interval is daily.

In some embodiments of any one of the kits provided herein, the at least one biomarker is selected from the group consisting of resting heart rate, blood oxygen level, systolic blood pressure, diastolic blood pressure, basal body temperature, blood sugar concentration, height, weight, body fat percentage, lean muscle mass percentage, body mass index, blood type, and resting metabolic rate.

In some embodiments of any one of the kits provided herein, the condition is characterized by the second value that is closer to a target value of the at least one biomarker as compared to the first value.

In some embodiments of any one of the kits provided herein, the condition is characterized by the second value that is further away from a target value of the at least one biomarker as compared to the first value.

In some embodiments of any one of the kits provided herein, the condition is characterized by the second value that is within a predetermined range around a target value of the at least one biomarker. In some embodiments, the first value is not within the predetermined range. In some embodiments, the first value is within the predetermined range.

In some embodiments of any one of the kits provided herein, the instructions further comprising, subsequent to (f): (1) applying the cannabinoid composition in an additional fixed volume to the region of the mouth of the individual; (2) subsequent to (1), measuring an additional value of the at least one biomarker for the individual; and (3) repeating (1) and (2) until the additional value meets the condition. In some embodiments, the second fixed volume and the additional fixed volume are substantially the same. In some embodiments, the second fixed volume and the additional fixed volume are different.

In some embodiments of any one of the kits provided herein, the instructions comprise performing (a) prior to (b).

Another aspect of the present disclosure provides a method for determining a target dosage of a cannabinoid composition, comprising any method instructed by the instructions of any one of the kits provided herein. In some embodiments, the method comprises: (a) measuring a first value of at least one biomarker for an individual; (b) applying the cannabinoid composition in a first fixed volume to a region of a mouth of the individual; (c) determining a first taste for the cannabinoid composition, wherein the first taste is characterized by one or more members selected from the group consisting of salty, bitter, sweet, sour, and savory; (d) applying the cannabinoid composition in a second fixed volume to the region of the mouth of the individual; (e) determining a second taste for the cannabinoid composition, wherein the second taste is characterized by one or more members selected from the group consisting of salty, bitter, sweet, sour, and savory; and (f) upon detecting a change between characterization of the second taste and the first taste, (1) measuring a second value of the at least one biomarker for the individual, and (2) determining, if the second value meets a condition, that a cumulative volume of the cannabinoid composition applied to the region of the individual is indicative of the target dosage of the cannabinoid composition for the individual.

Another aspect of the present disclosure provides a kit comprising: a cannabinoid composition; an applicator device that dispenses the cannabinoid composition in a fixed volume; and instructions for determining a target dosage according to a method of: (a) using the applicator device to apply the cannabinoid composition in a first fixed volume to a region of a mouth of the individual; (b) determining a first taste for the cannabinoid composition, wherein the first taste is characterized by one or more members selected from the group consisting of: salty, bitter, sweet, sour, and savory; (c) applying the cannabinoid composition in a second fixed volume to the region of the mouth of the individual; (d) determining a second taste for the cannabinoid composition, wherein the second taste is characterized by one or more members selected from the group consisting of: salty, bitter, sweet, sour, and savory; and (e) upon detecting a change between characterization of the second taste and the first taste, determining that a cumulative volume of the cannabinoid composition applied to the region of the individual is indicative of the target dosage of the cannabinoid composition for the individual.

In some embodiments of any one of the kits provided herein, the applicator device is configured to dispense the cannabinoid composition in an additional fixed volume, wherein the fixed volume and the additional fixed volume are different.

In some embodiments of any one of the kits provided herein, the instructions for determining the target dosage comprises instructions for repeating (c) through (d) until the change is detected to determine the cumulative volume.

In some embodiments of any one of the kits provided herein, the cannabinoid composition comprises cannabidiol. In some embodiments of any one of the kits provided herein, the cannabinoid composition comprises tetrahydrocannabinol at less than 0.3% by weight.

In some embodiments of any one of the kits provided herein, the first fixed volume is between about 1 milliliter (mL) to about 5 mL. In some embodiments of any one of the kits provided herein, the first fixed volume and the second fixed volume are substantially the same. In some embodiments of any one of the kits provided herein, the second fixed volume is less than the first fixed volume.

In some embodiments of any one of the kits provided herein, the instructions for determining the target dosage comprises instructions for rinsing the mouth. In some embodiments, the instructions for determining the target dosage comprises instructions for rinsing prior to (a). In some embodiments, the instructions for determining the target dosage comprises instructions for rinsing prior to (c). In some embodiments, the instructions for determining the target dosage comprises instructions for rinsing subsequent to (a).

In some embodiments of any one of the kits provided herein, the region of the mouth comprises the sublingual region. In some embodiments of any one of the kits provided herein, the region of the mouth comprises the ventral region of the tongue.

In some embodiments of any one of the kits provided herein, the instructions comprise characterizing the first taste by at least bitter. In some embodiments of any one of the kits provided herein, the instructions comprise characterizing the second taste by at least sweet.

In some embodiments of any one of the kits provided herein, the instructions comprise determining a second dosage of the cannabinoid composition at a fixed interval. In some embodiments, the fixed interval is daily.

Another aspect of the present disclosure provides a method for determining a target dosage of a cannabinoid composition, comprising any method instructed by the instructions of any one of the kits provided herein. In some embodiments, the method comprises: (a) applying the cannabinoid composition in a first fixed volume to a region of a mouth of the individual; (b) determining a first taste for the cannabinoid composition, wherein the first taste is characterized by one or more members selected from the group consisting of: salty, bitter, sweet, sour, and savory; (c) applying the cannabinoid composition in a second fixed volume to the region of the mouth of the individual; (d) determining a second taste for the cannabinoid composition, wherein the second taste is characterized by one or more members selected from the group consisting of salty, bitter, sweet, sour, and savory; and (e) upon detecting a change between characterization of the second taste and the first taste, determining that a cumulative volume of the cannabinoid composition applied to the region of the individual is indicative of the target dosage of the cannabinoid composition for the individual.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts a schematic representation of a method for determining an approximate target dosage of a cannabinoid composition.

FIG. 2 depicts a schematic representation of a method for determining a precise target dosage of a cannabinoid composition.

FIG. 3 illustrates the components of a computer system that may be used to assist in the determination of a target dosage of a cannabinoid composition.

FIG. 4 shows a graphical depiction of a dosage vs. efficacy graph for a microencapsulated cannabinoid composition with multiple target dosage volumes.

FIG. 5 shows an example kit for determining a target dosage of a cannabinoid composition for an individual.

### DETAILED DESCRIPTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

The present disclosure provides methods and kits for determining an individual's optimal or target dosage of a cannabinoid composition. The methods and kits may comprise sublingual dosing of metered quantities of a liquid that contain one or more cannabinoid compounds. The sublingual dosing methods allow for an accurate determination of the optimal or target dosage for a given individual at a given time. The described dosing method may be based upon changes in taste or flavor of a cannabinoid composition when applied to a region of the mouth such as the sublingual region.

The present disclosure also provides methods and kits for predicting an optimal or target cannabinoid dosage via the correlation of oral dosing with various other biomarkers. The methods and kits described herein may monitor and track changes in optimal or target cannabinoid dosage of a given individual with time, and correlate the changes in cannabinoid dosage to changes in other biomarkers, such as height, weight, blood pressure, resting heart rate, and blood sugar level of the given individual. In some instances, computerized algorithms may be implemented to monitor and predict an individual's optimal or target dosage based upon historical dosage and biomarker data. Beneficially, such methods and kits for determining target dosage may facilitate efficient usage of a cannabinoid composition, such that minimal volumes of said composition are expended to achieve an efficacy (e.g., therapeutic efficacy) of the cannabinoid composition to optimize resources and prevent overdosage, as well as ensure that the efficacy is achieved.

Cannabinoid Compositions

Cannabinoids comprise a class of chemical compounds that bind to the cannabinoid receptor system of many animals, including humans. Cannabinoids may be broadly grouped into categories such as endocannabinoids that are naturally-produced by animals for internal signaling, phytocannabinoids that are produced by plants, and synthetic cannabinoids that are manufactured. Cannabinoids may produce a broad range of pharmacological effects, making them an active target for pharmaceutical research. Most commercially available cannabinoids are derived from plants of the Cannabis genus. At least 100 cannabinoid compounds have been derived from cannabis plants, including, for example, compounds such as tetrahydrocannabinol (THC), cannabinol (CBN), and cannabidiol (CBD).

Cannabinoids disclosed herein include but are not limited to cannabigerol-type (CBG), cannabigerolic acid (CBGA), cannabigerolic acid monomethylether (CBGAM), cannabigerol monomethyl ether (CBGM), cannabichromene-type (CBC), cannabichromanon (CBCN), cannabichromenic acid (CBCA), cannabichromevarin-type (CBCV), cannabichromevarinic acid (CBCVA), cannabidiol-type (CBD), tetrahydrocannabinol-type (THC), iso-tetrahydrocannabinol-type (iso-THC), cannabinol-type (CBN), cannabinolic acid (CBNA), cannabinol methylether (CBNM), cannabinol-C₄ (CBN-C₄), cannabinol-C₂ (CBN-C₂), cannabiorcol (CBN-C₁), cannabinodiol (CBND), cannabielsoin-type (CBE), cannabielsoic acid A (CBEA-A), cannabielsoic acid B (CBEA-B), cannabicyclol-type (CBL), cannabicyclolic acid (CBLA), cannabicyclovarin (CBLV), cannabicitran-type (CBT), cannabitriol, cannabitriolvarin (CBTV), ethoxy-cannabitiolvarin (CBTVE), cannabivarin-type (CBV), cannabinodivarin (CBVD), tetrahydrocannabivarin-type (THCV), cannabidivarin-type (CBDV), cannabigerovarin-type (CBGV), cannabigerovarinic acid (CBGVA), cannabifuran (CBF), dehydrocannabifuran (DCBF), and cannabiripsol (CBR) cannabinoids.

The cannabinoids of the subject compositions disclosed herein can comprise cannabidiol-class compounds, including but not limited to cannabidiol (CBD), cannabidiolic acid (CBDA), cannabidiol monomethylether (CBDM), cannabidiol-C₄ (CBD-C₄), cannabidivarin (CBDV), cannabidivarinic acid (CBDVA), cannabidiorcol (CBD-C₁), and combinations thereof. CBD can comprise delta-1-cannabidiol, delta-2-cannabidiol, delta-3-cannabidiol, delta-3,7-cannabidiol, delta-4-cannabidiol, delta-5-cannabidiol, delta-6-cannabidiol, and combinations thereof.

The compositions of the present disclosure can comprise tetrahydrocannabinol (THC) as a type of cannabinoids. THC can comprise delta-9-THC, delta-8-THC, and combinations thereof. THC can comprise delta-6a,7-tetrahydrocannabinol, delta-7-tetrahydrocannabinol, delta-8-tetrahydrocannabinol, delta-9,11-tetrahydrocannabinol, delta-9-tetrahydrocannabinol, delta-10-tetrahydrocannabinol, delta-6a,10a-tetrahydrocannabinol, and combinations thereof. Delta-9-tetrahydrocannabinol can comprise stereoisomers including (6aR,10aR)-delta-9-tetrahydrocannabinol, (6aS,10aR)-delta-9-tetrahydrocannabinol, (6aS,10aS)-delta-9-tetrahydrocannabinol, (6aR,10aS)-delta-9-tetrahydrocannabinol, and combinations thereof.

The cannabinoid compositions described herein may also contain other ingredients, including terpenes, herbal ingredients, essential oils, and other ingredients.

The cannabinoid compositions of the present disclosure can comprise one or more terpene compounds, including but not limited to terpenoids such as monoterpenoids, sesquiterpenoids, diterpenoids, and triterpenoids. Terpenes can be acyclic, monocyclic, or polycyclic. Terpenes can include but are not limited to myrcene, limonene, linalool, trans-ocimene, cis-ocimene, alpha-pinene, beta-pinene, alpha-humulene (alpha-caryophyllene), beta-caryophyllene, delta-3-carene, trans-gamma-bisabolene, cis-gamma-bisabolene, trans-alpha-famesene, cis-beta-farnesene, beta-fenchol, beta-phellandrene, guajol, alpha-gualene, alpha-eudesmol, beta-eudesmol, gamma-eudesmol, terpinolene, alpha-selinene, beta-selinene, alpha-terpineol, fenchone, camphene, cis-sabinene hydrate, alpha-trans-bergamotene, alpha-cis-bergamotene, borneol, gamma-curcumene, alpha-thujene, epi-alpha-bisabolol, ipsdienol, alpha-ylangene, beta-elemene, gamma-muurolene, alpha-cadinene, alpha-longipinene, caryophyllene oxide, and combinations thereof.

The cannabinoid compositions of the present disclosure can comprise one or more herbal ingredients. Herbal ingredients can include but are not limited to: maca, he shou wu, iporuru (Alchornea castaneifolia), kanna (Sceletium Tortosum), honokiol (Magnolia grandiflora), Sour Jujube Seed Semen (Ziziphi Spinosae), Cnidium Fruit (Fructus Cnidii), Corydalis Rhizome (Corydalis yanhusuo), Albizia Bark or Flower (Cortex albiziae), Ginseng (Panax ginseng), Polygonum (Polygoni Multiflori), Fu ling (Poria cocos), Comus Fruit (Fructus corni), Chinese Yam (Rhizoma dioscoreae), Muira puama, Dendrobium sp., Licorice Root Radix (Glycyrrhizae Preparata), Cordyceps (Cordyceps sinensis), Chinese Angelica Root (Angelicae Sinensis), Kratom (Mitragyna speciosa), Bacopa monnieri, Catuaba, Ashwaghanda, Peganum harmala, Wheat Grass, Alfalfa Grass, Oat Grass, Kamut Grass, Echinacea, Chlorella, Amla Fruit, Stinging Nettles, Carob, Mesquite, Chuchuhuasai, Clavo Huasca, Chanca Piedra, Guayusa Powder, Rhodiola rosea, Shilajit, Higenamine, Moringa (Moringa oleifera), Horny Goat Weed (Epidmedium), Astragalus, Aloe Vera, Turmeric, Pine Pollen, Cucurmine (tumeric compound), Hops, Xanthohumol (hops compound), Passion Flower, Mucuna Puriens, Tusli, Black Pepper, Bioperine (black pepper compound), Siberian Ginseng, American Ginseng, Yerba Mate, Lemon Balm, Astragulus, Kava Kava, Schizandra, Skullcap, Valerian, California Poppy, Epidmedium, Pau D'Arco, Gingko, Blue Lotus, White Lilly, and Cacao. Herbal ingredients can comprise essential oils.

Example essential oils include but are not limited to: Linalool; B-Caryophyllene; B-Myrcene; D-Limonene; Humulene; a-Pinene; Ylang Ylang (Cananga odorata); Yarrow (Achillea millefolium); Violet (Viola odorata); Vetiver (Vetiveria zizanoides); Vanilla (Vanilla plantifolia); Tuberose (Polianthes tuberosa); Thyme (Thymus vulgaris L.); Tea Tree (Melaleuca alternifolia); Tangerine (Citrus reticulata); Spruce, Black (Picea mariana); Spruce (Tsuga Canadensis); Spikenard (Nardostachys jatamansi); Spearmint (Mentha spicata); Sandalwood (Santalum spicatum); Rosewood (Aniba rosaeodora); Rosemary Verbenone (Rosmarinus officinalis); Rosemary (Rosmarinus officinalis); Rose (Rosa damascena); Rose Geranium (Pelargonium roseum); Ravensara (Ravensara aromatica); Plai (Zingiber cassumunar) Pine Needle (Pinus sylvestris L.) Petitgrain (Citrus aurantium); Peppermint (Mentha piperita); Pepper, Black (Piper nigrum L.); Patchouli (Pogostemon cablin); Palo Santo (Bursera graveolens); Palmarosa (Cymbopogon martini); Osmanthus (Osmanthus fragrans); Oregano (Origanum vulgare); Orange, Sweet (Citrus sinensis); Oak Moss (Evemia prunastri); Nutmeg (Myristica fragrans) Niaouli (Melaleuca viridifloria); Neroli (aka Orange Blossom) (Citrus aurantium); Myrtle (Myrtus communis); Myrrh (Commiphora myrrha); Mimosa (Acacia decurrens); Melissa (Melissa officinalis L.); Marjoram, Sweet (Origanum majorana); Manuka (Leptospermum scoparium); Mandarin, Red (Citrus deliciosa); Mandarin (Citrus deliciosa); Lotus, White (Nelumbo nucifera); Lotus, Pink (Nelumbo nucifera); Lotus, Blue (Nelumbo nucifera); Lime (Citrus aurantifolia); Lily (Lilum aurantum); Lemongrass (Cymbopogon citratus); Lemon (Citrus limonum); Lavender (Lavandula angustifolium); Lavandin (Lavandula hybrida grosso); Kanuka (Kunzea ericoides); Juniper Berry (Juniperus cummunis); Jasmine (Jasminum officinale); Jasmine Abs (Jasminum sambac); Helichrysum (Helichrysum italicum); Grapefruit, White (Citrus x paradisi); Grapefruit, Pink (Citrus paradisi); Ginger (Zingiber officinalis); Geranium (Pelargonium graveolens); Geranium, Bourbon (Pelargonium graveolens, 'Herit); Gardenia (Gardenia jasminoides); Galbanum (Ferula galbaniflua); Frankincense (Boswellia carterii); Frangipani (Plumeria alba); Fir Needle White (Abies alba); Fir Needle Siberia (Abies siberica); Fir Needle Canada (Abies balsamea); Fennel, Sweet (Foeniculum vulgare); Eucalyptus Smithii. Eucalyptus Radiata, Eucalyptus Globulus, Eucalyptus Citriodora, Eucalyptus Blue Mallee (Eucalyptus polybractea); Elemi (Canarium luzonicum); Dill (Anethum graveolens); Cypress (Cupressus sempervirens); Cumin (Cuminum cyminum); Coriander (Coriandum sativum); Cocoa (Theobroma cacao); Clove (Eugenia caryophylatta); Clary Sage (Salvia sclarea); Cistus (aka Labdanum) (Cistus ladaniferus L.); Cinnamon (Cinnamomum zeylanicum); Chamomile, Roman (Anthemis nobilis); Chamomile, Blue (Matricaria chamomilla); Celery Seed (Apium graveolins); Cedarwood, Western Red (Thuja plicata); Cedarwood, Blood (Juniperus virginiana); Cedarwood Atlas (Cedrus atlantica); Carrot Seed (Daucus carota); Cardamon (Elettaria cardamomum); Caraway Seed (Carum carvi); Cajeput (Melaleuca cajuputi); Cade (Juniperus oxycedrus); Birch, White (Betula alba); Birch, Sweet (Betula lenta); Bergamot (Citrus bergamia); Bay Laurel (Laurus nobilis); Basil (Ocimum basilicum); Basil, Holy (Ocimum sanctum); Basil (Ocimum basilicum); Balsam Poplar (Populus balsamifera); Balsam Peru (Myroxylon balsamum); Angelica (Angelica archangelica L.); and combinations thereof.

Cannabinoid compositions of the present disclosure can comprise one or more additional compounds or derivatives thereof, including but not limited to pregnenolone or other compounds that counteract THC intoxication, MSM, fulvic acid, L-Theanine, Fish Oil, and phenylethylamine (PEA).

In some examples, a cannabinoid composition of the present disclosure comprises ingredients including tulsi, lemon balm, passion flower, and blue lotus. In some examples, a cannabinoid composition of the present disclosure comprises ingredients including cacao, maca, schizandra, and Siberian ginseng. In some examples, a cannabinoid composition of the present disclosure comprises ingredients including kava, skullcap, valerian, hops, and California poppy. In some examples, a cannabinoid composition of the present disclosure comprises ingredients including maca, catuba, epidmedium, and pao d'arco. In some examples, a cannabinoid composition of the present disclosure comprises ingredients including ashwaganda, ginko, and albiza. In some examples, a cannabinoid composition of the present disclosure comprises ingredients including reishi, lion's mane, maitake, and chaga. In some examples, a cannabinoid composition of the present disclosure comprises ingredients including MSM, vitamin C, turmeric, CBD oil, bioperine, and xanthohumol.

The cannabinoid compositions of the present disclosure can comprise pregnenolone, including derivatives thereof. Pregnenolone can help protect a subject from cannabis intoxication, for example from THC. Pregnenolone or derivatives thereof can be formulated to be water soluble. A cannabinoid composition of the present disclosure can comprise between about 1 and 50 milligrams (mg) of pregnenolone or derivatives thereof. For example, a unit dosage of the present disclosure can comprise between about 1 and 50 milligrams (mg) of pregnenolone. Cannabinoid compositions of the present disclosure (e.g., unit dosages) can comprise about 1, 2, 3, 4, 5, 6, 7, 8, 9, 10, 15, 20, 25, 30, 35, 40, 45, or 50 mg of pregnenolone. Cannabinoid compositions of the present disclosure (e.g., unit dosages) can comprise at least about 1, 2, 3, 4, 5, 6, 7, 8, 9, 10, 15, 20, 25, 30, 35, 40, 45, or 50 mg of pregnenolone. Cannabinoid compositions of the present disclosure (e.g., unit dosages) can comprise at most about 1, 2, 3, 4, 5, 6, 7, 8, 9, 10, 15, 20, 25, 30, 35, 40, 45, or 50 mg of pregnenolone. Cannabinoid compositions comprising pregnenolone can be used in combination with any other compounds, ingredients, or formulations described herein, including esters, cyclodextrin complexes, microcapsules (e.g., sodium alginate microcapsules), immediate release formulations, delayed or extended release formulations, transbuccal formulations, and sublingual formulations. Alternatively or in addition, cannabinoid compositions of the present disclosure may comprise one or more other compounds that counteract cannabis intoxication (e.g., THC intoxication).

Cannabinoids may be produced by extractive methods or synthetic routes. In some aspects, cannabinoids may be directly extracted from cannabis plants via solvent methods. Extracted cannabinoids may undergo other chemical conversions or separations to affect the relative amounts of cannabinoids within a cannabinoid composition. In some aspects, a cannabinoid composition may be formulated to have increased levels of cannabinoids such as CBD and CBN and decreased levels of cannabinoids such as THC. In some aspects, a cannabinoid composition may be processed to chemically convert certain cannabinoids, e.g., the conversion of THC to CBN via exposure to UV light. Cannabinoid compositions may also contain other natural or synthetic compounds, such as terpenes. Terpenes may be co-extracted as a natural chemical constituent of a cannabis plant or may be blended from the extracts of other plants.

Cannabinoid extractions may comprise any method that selectively removes the cannabinoid content of cannabis biomass while retaining as much of the solid structure of the biomass as possible. Compounds used in compositions of the present disclosure can be extracted by a variety of methods. For example, extraction can be performed by maceration, infusion, decoction, percolation, Soxhlet extraction, counter current extraction, or ultrasonication. Cannabinoid extractions may include solvent extractions and supercritical CO₂ extractions. Cannabinoid extractions may be performed in batch or continuous processes. Cannabinoid extractions may require one or more extraction devices. Cannabinoid extractions may include ancillary components such as distillation devices, liquid-liquid separation units, chromatography units, pumps, compressors, chillers, and heaters. Cannabinoid extraction processes may be carefully controlled for temperature, pressure, and residence time of the biomass material in the extraction device.

Solvent extractions may be performed on various cannabis-derived biomass components, including seeds, trichomes, leaves, stems, and buds. Solvents may comprise ethanol, propane, and butane. Solvent extraction may be performed at a broad range of temperatures depending upon the desired outcome of the extraction process. Solvent extraction may be performed at a temperature of about −80 degrees Celsius (° C.), −70° C., −60° C., −50° C., −40° C., −30° C., −20° C., −10° C., 0° C., 10° C., 20° C., 30° C., 40° C., 50° C., 60° C., 70° C., or about 80° C. Solvent extraction may be performed at a temperature that is at least about −80° C., −70° C., −60° C., −50° C., −40° C., −30° C., −20° C., −10° C., 0° C., 10° C., 20° C., 30° C., 40° C., 50° C., 60° C., 70° C., 80° C., or more. Solvent extraction may be performed at a temperature that is at most about 80° C., 70° C., 60° C., 50° C., 40° C., 30° C., 20° C., 10° C., 0° C., −10° C., −20° C., −30° C., −40° C., −50° C., −60° C., −70° C., −80° C., or less. Solvent extraction may occur in a temperature range from about −80° C. to about −60° C., about −80° C. to about −40° C., about −80° C. to about −20° C., about −80° C. to about 0° C., about −80° C. to about 20° C., about −80° C. to about 40° C., about −80° C. to about 60° C., about −80° C. to about 80° C., about −60° C. to about −40° C., about −60° C. to about −20° C., about −60° C. to about 0° C., about −60° C. to about 20° C., about −60° C. to about 40° C., about −60° C. to about 60° C., about −60° C. to about 80° C., about −40° C. to about −20° C., about −40° C. to about 0° C., about −40° C. to about 20° C., about −40° C. to about 40° C., about −40° C. to about 60° C., about −40° C. to about 80° C., about −20° C. to about 0° C., about −20° C. to about 20° C., about −20° C. to about 40° C., about −20° C. to about 60° C., about −20° C. to about 80° C., about 0° C. to about 20° C., about 0° C. to about 40° C., about 0° C. to about 60° C., about 0° C. to about 80° C., about 20° C. to about 40° C., about 20° C. to about 60° C., about 20° C. to about 80° C., about 40° C. to about 60° C., about 40° C. to about 80° C., or about 60° C. to about 80° C.

Solvent extraction of cannabinoids may be performed at about atmospheric pressure. Solvent extraction of cannabinoids may be performed at a pressure above atmospheric pressure. A solvent extraction may be performed at a pressure of about 2 bar, 3 bar, 4 bar, 5 bar, 10 bar, 20 bar, 40 bar, 60 bar, 80 bar, 100 bar, 150 bar, or about 200 bar. A solvent extraction of cannabinoids may be performed at a pressure of at least about 2 bar, 3 bar, 4 bar, 5 bar, 10 bar, 20 bar, 40 bar, 60 bar, 80 bar, 100 bar, 150 bar, 200 bar, or more. A solvent extraction of cannabinoids may be performed at a pressure of at most about 200 bar, 150 bar, 100 bar, 80 bar, 60 bar, 40 bar, 20 bar, 10 bar, 5 bar, 4 bar, 3 bar, 2 bar, or less. A solvent extraction may have a variable pressure with a pressure range from about 1 bar to about 2 bar, 1 bar to about 5 bar, 1 bar to about 10 bar, 1 bar to about 20 bar, 1 bar to about 40 bar, 1 bar to about 60 bar, 1 bar to about 80 bar, 1 bar to about 100 bar, 1 bar to about 150 bar, 1 bar to about 200 bar, 2 bar to about 5 bar, 2 bar to about 10 bar, 2 bar to about 20 bar, 2 bar to about 40 bar, 2 bar to about 60 bar, 2 bar to about 80 bar, 2 bar to about 100 bar, 2 bar to about 150 bar, 2 bar to about 200 bar, 5 bar to about 10 bar, 5 bar to about 20 bar, 5 bar to about 40 bar, 5 bar to about 60 bar, 5 bar to about 80 bar, 5 bar to about 100 bar, 5 bar to about 150 bar, 5 bar to about 200 bar, 10 bar to about 20 bar, 10 bar to about 40 bar, 10 bar to about 60 bar, 10 bar to about 80 bar, 10 bar to about 100 bar, 10 bar to about 150 bar, 10 bar to about 200 bar, 20 bar to about 40 bar, 20 bar to about 60 bar, 20 bar to about 80 bar, 20 bar to about 100 bar, 20 bar to about 150 bar, 20 bar to about 200 bar, 40 bar to about 60 bar, 40 bar to about 80 bar, 40 bar to about 100 bar, 40 bar to about 150 bar, 40 bar to about 200 bar, 60 bar to about 80 bar, 60 bar to about 100 bar, 60 bar to about 150 bar, 60 bar to about 200 bar, 80 bar to about 100 bar, 80 bar to about 150 bar, 80 bar to about 200 bar, 100 bar to about 150 bar, 100 bar to about 200 bar, or about 150 bar to about 200 bar.

A solvent extraction process for cannabinoids from cannabis biomass may occur for a residence time of about 1 minutes (min), 5 mins, 10 mins, 30 mins, 60 mins, 2 hours, 4 hours, 6 hours, or about 12 hours. A solvent extraction process for cannabinoids from cannabis biomass may occur for a residence time of at least about 1 min, 5 mins, 10 mins, 30 mins, 60 mins, 2 hours, 4 hours, 6 hours, 12 hours, 24 hours, or more. A solvent extraction process for cannabinoids from cannabis biomass may occur for a residence time of at most about 24 hours, 12 hours, 6 hours, 4 hours, 2 hours, 60 mins, 30 mins, 10 mins, 5 mins, 1 min, or less.

Supercritical carbon dioxide (CO₂) extractions may be performed on various cannabis-derived biomass components, including seeds, trichomes, leaves, stems, and buds. Additional solvents, such as ethanol, may be employed depending upon the desired outcome of the extraction. Supercritical CO₂ extraction may be performed at a broad range of temperatures depending upon the desired outcome of the extraction process. Supercritical CO₂ extraction will always occur at a temperature and pressure above the critical point of CO₂, namely 31° C. and 74 bar. Supercritical CO₂ extraction may be performed at a temperature of about 31° C., 40° C., 50° C., 60° C., 70° C., 80° C., 90° C., 100° C., 110° C., 120° C., 130° C., 140° C., or about 150° C. Supercritical CO₂ extraction may be performed at a temperature that is at least about 31° C., 40° C., 50° C., 60° C., 70° C., 80° C., 90° C., 100° C., 110° C., 120° C., 130° C., 140° C., 150° C., or more. Supercritical CO₂ extraction may be performed at a temperature that is at most about 150° C., 140° C., 130° C., 120° C., 110° C., 100° C., 90° C., 80° C., 70° C., 60° C., 50° C., 40° C., 31° C., or less. Supercritical CO₂ extraction may occur in a temperature range from about 31° C. to about 50° C., about 31° C. to about 70° C., about 31° C. to about 90° C., about 31° C. to about 110° C., about 31° C. to about 130° C., about 31° C. to about 150° C., about 50° C. to about 70° C., about 50° C. to about 90° C., about 50° C. to about 110° C., about 50° C. to about 130° C., about 50° C. to about 150° C., about 70° C. to about 90° C., about 70° C. to about 110° C., about 70° C. to about 130° C., about 70° C. to about 150° C., about 90° C. to about 110° C., about 90° C. to about 130° C., about 90° C. to about 150° C., about 110° C. to about 130° C., about 110° C. to about 150° C., or about 130° C. to about 150° C.

Supercritical CO₂ extraction of cannabinoids may be performed at a pressure above atmospheric pressure. A supercritical CO₂ extraction may be performed at a pressure of about 74 bar, 80 bar, 100 bar, 120 bar, 140 bar, 160 bar, 180 bar, 200 bar, 250 bar, 300 bar, 350 bar, 400 bar, 450 bar, or about 500 bar. A supercritical CO₂ extraction of cannabinoids may be performed at a pressure of at least about 74 bar, 80 bar, 100 bar, 120 bar, 140 bar, 160 bar, 180 bar, 200 bar, 250 bar, 300 bar, 350 bar, 400 bar, 450 bar, 500 bar, or more. A supercritical CO₂ extraction of cannabinoids may be performed at a pressure of at most about 500 bar, 450 bar, 400 bar, 350 bar, 300 bar, 250 bar, 200 bar, 180 bar, 160 bar, 140 bar, 120 bar, 100 bar, 80 bar, o 74 bar, or less. A supercritical CO₂ extraction may have a variable pressure with a pressure range from about 74 bar to about 80 bar, about 74 bar to about 100 bar, about 74 bar to about 120 bar, about 74 bar to about 80 bar, about 74 bar to about 140 bar, about 74 bar to about 160 bar, about 74 bar to about 180 bar, about 74 bar to about 200 bar, about 74 bar to about 300 bar, about 74 bar to about 400 bar, about 74 bar to about 500 bar, about 80 bar to about 100 bar, about 80 bar to about 120 bar, about 80 bar to about 80 bar, about 80 bar to about 140 bar, about 80 bar to about 160 bar, about 80 bar to about 180 bar, about 80 bar to about 200 bar, about 80 bar to about 300 bar, about 80 bar to about 400 bar, about 80 bar to about 500 bar, about 100 bar to about 120 bar, about 100 bar to about 80 bar, about 100 bar to about 140 bar, about 100 bar to about 160 bar, about 100 bar to about 180 bar, about 100 bar to about 200 bar, about 100 bar to about 300 bar, about 100 bar to about 400 bar, about 100 bar to about 500 bar, about 120 bar to about 140 bar, about 120 bar to about 160 bar, about 120 bar to about 180 bar, about 120 bar to about 200 bar, about 120 bar to about 300 bar, about 120 bar to about 400 bar, about 120 bar to about 500 bar, about 140 bar to about 160 bar, about 140 bar to about 180 bar, about 140 bar to about 200 bar, about 140 bar to about 300 bar, about 140 bar to about 400 bar, about 140 bar to about 500 bar, about 160 bar to about 180 bar, about 160 bar to about 200 bar, about 160 bar to about 300 bar, about 160 bar to about 400 bar, about 160 bar to about 500 bar, about 180 bar to about 200 bar, about 180 bar to about 300 bar, about 180 bar to about 400 bar, about 180 bar to about 500 bar, about 200 bar to about 300 bar, about 200 bar to about 400 bar, about 200 bar to about 500 bar, about 300 bar to about 400 bar, about 300 bar to about 500 bar, or from about 400 bar to about 500 bar.

A supercritical CO₂ extraction process for cannabinoids from cannabis biomass may occur for a residence time of about 1 min, 5 mins, 10 mins, 30 mins, 60 mins, 2 hours, 4 hours, 6 hours, or about 12 hours. A supercritical CO₂ extraction process for cannabinoids from cannabis biomass may occur for a residence time of at least about 1 min, 5 mins, 10 mins, 30 mins, 60 mins, 2 hours, 4 hours, 6 hours, 12 hours, 24 hours, or more. A supercritical CO₂ extraction process for cannabinoids from cannabis biomass may occur for a residence time of at most about 24 hours, 12 hours, 6 hours, 4 hours, 2 hours, 60 mins, 30 mins, 10 mins, 5 mins, 1 min, or less.

An extraction process may comprise an additional process, method, device, or component that allows the cannabinoid composition of the extracted oil to be altered. In some aspects, an alteration may comprise removing or diluting the concentration of THC relative to the concentration of CBD. In other aspects, an alteration may comprise converting THC to CBN or another cannabinoid. The additional process, method, device, or component may be employed until the concentration of THC drops to a prescribed level in the extracted oil. An extracted oil may have a THC concentration of less than about 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or less than about 3.0% on a weight basis. An extracted oil may have a THC concentration of about 0.01% to about 0.05%, about 0.01% to about 0.1%, about 0.01% to about 0.3%, about 0.01% to about 0.5%, about 0.01% to about 1.0%, about 0.01% to about 1.5%, about 0.01% to about 2.0%, about 0.01% to about 2.5%, about 0.01% to about 3.0%, about 0.05% to about 0.1%, about 0.05% to about 0.3%, about 0.05% to about 0.5%, about 0.05% to about 1.0%, about 0.05% to about 1.5%, about 0.05% to about 2.0%, about 0.05% to about 2.5%, about 0.05% to about 3.0%, about 0.1% to about 0.3%, about 0.1% to about 0.5%, about 0.1% to about 1.0%, about 0.1% to about 1.5%, about 0.1% to about 2.0%, about 0.1% to about 2.5%, about 0.1% to about 3.0%, about 0.3% to about 0.5%, about 0.3% to about 1.0%, about 0.3% to about 1.5%, about 0.3% to about 2.0%, about 0.3% to about 2.5%, about 0.3% to about 3.0%, about 0.5% to about 1.0%, about 0.5% to about 1.5%, about 0.5% to about 2.0%, about 0.5% to about 2.5%, about 0.5% to about 3.0%, about 1.0% to about 1.5%, about 1.0% to about 2.0%, about 1.0% to about 2.5%, about 1.0% to about 3.0%, about 1.5% to about 2.0%, about 1.5% to about 2.5%, about 1.5% to about 3.0%, about 2.0% to about 2.5%, about 2.0% to about 3.0%, or about 2.5% to about 3.0%.

In one aspect, an extraction process unit may comprise a chamber for the dilution of THC from the extract oil. The dilution chamber may comprise a secondary solvent that preferentially solvates THC over CBD. The extraction unit may be stirred or agitated to increase contact time between the two solvents, enhancing the extraction of THC into the secondary solvent phase. In another aspect, an extraction process unit may be operatively connected to a second separation unit comprising an adsorbent or chromatographic medium that preferentially binds THC. Extraction may be a continuous process where the extraction solution is fed to consecutive units. Extraction may be a recycling process where extract oils are iteratively passed between an extraction unit and a THC removal unit.

In another aspect, an ultraviolet (UV) light chamber may be coupled to an extraction process unit. The UV light may irradiate the fluids within the extraction unit. The UV light may be configured to irradiate an exit port or a standalone unit that recycles fluids back to the extraction unit. The UV light may be of a sufficient wavelength, intensity, and residence time to convert some or all of the THC to CBN or perform other photochemical conversions. The wavelength of the light may be chosen to optimize a particular photochemical conversion. The UV light may have a wavelength of about 200 nanometers (nm), 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, or about 400 nm. The UV light may have a wavelength range comprising a wavelength of about 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, or about 400 nm. The UV light may have a range from about 200 nm to about 220 nm, about 200 nm to about 240 nm, about 200 nm to about 260 nm, about 200 nm to about 280 nm, about 200 nm to about 300 nm, about 200 nm to about 320 nm, about 200 nm to about 340 nm, about 200 nm to about 360 nm, about 200 nm to about 380 nm, about 200 nm to about 400 nm, about 220 nm to about 240 nm, about 220 nm to about 260 nm, about 220 nm to about 280 nm, about 220 nm to about 300 nm, about 220 nm to about 320 nm, about 220 nm to about 340 nm, about 220 nm to about 360 nm, about 220 nm to about 380 nm, about 220 nm to about 400 nm, about 240 nm to about 260 nm, about 240 nm to about 280 nm, about 240 nm to about 300 nm, about 240 nm to about 320 nm, about 240 nm to about 340 nm, about 240 nm to about 360 nm, about 240 nm to about 380 nm, about 240 nm to about 400 nm, about 260 nm to about 280 nm, about 260 nm to about 300 nm, about 260 nm to about 320 nm, about 260 nm to about 340 nm, about 260 nm to about 360 nm, about 260 nm to about 380 nm, about 260 nm to about 400 nm, about 280 nm to about 300 nm, about 280 nm to about 320 nm, about 280 nm to about 340 nm, about 280 nm to about 360 nm, about 280 nm to about 380 nm, about 280 nm to about 400 nm, about 300 nm to about 320 nm, about 300 nm to about 340 nm, about 300 nm to about 360 nm, about 300 nm to about 380 nm, about 300 nm to about 400 nm, about 320 nm to about 340 nm, about 320 nm to about 360 nm, about 320 nm to about 380 nm, about 320 nm to about 400 nm, about 340 nm to about 360 nm, about 340 nm to about 380 nm, about 340 nm to about 400 nm, about 360 nm to about 380 nm, about 360 nm to about 400 nm, or about 380 nm to about 400 nm.

Extracted oils may have a residence time of UV exposure of at least about 1 second, 5 seconds, 10 seconds, 20 seconds, 30 seconds, 1 minute, 5 minutes, 10 minutes, 30 minutes, 60 minutes, 2 hours, 3 hours, 6 hours, 10 hours, or more. Extracted oils may have a residence time for UV exposure of at most about 10 hours, 6 hours, 3 hours, 2 hours, 60 minutes, 30 minutes, 10 minutes, 5 minutes, 1 minute, 30 seconds, 20 seconds, 10 seconds, 5 seconds, 1 second, or less.

Cannabinoid extracts may be formulated into a cannabinoid composition by blending the cannabinoid extracts with other chemical constituents, including, but not limited to, other plant extracts, oils, and alcohols. Cannabinoid extracts may be combined with various oils including hempseed oil, coconut oil, olive oil, vegetable oil, canola oil, peanut oil, walnut oil, almond oil, cashew oil, sesame oil, palm oil, avocado oil, rapeseed oil, grapeseed oil, corn oil, soybean oil, fish oil, and flax oil. Cannabinoids may be combined with alcohols, such as azeoptropically-distilled ethanol to form cannabinoid tinctures.

A cannabinoid oil or cannabinoid tincture may comprise a microencapsulated form of the cannabinoid composition. Cannabinoids may be encapsulated in liposomes or colloids. A liposome may comprise a lipid bilayer with cannabinoids and other chemical constituents encapsulated within the hydrophobic region of the liposome. A colloid may comprise a spherical lipid monolayer with cannabinoids and other chemical constituents encapsulated within the hydrophobic region of the colloid. Cannabinoid compositions described herein may be microencapsulated in an oil or tincture to create a cannabinoid product with a possibly higher bioavailability. Alternatively or in addition, a cannabinoid oil or cannabinoid tincture may comprise the cannabinoid composition in non-encapsulated form.

Microencapsulation can be performed with a microencapsulation device, including microfluidic droplet generation or encapsulation devices. An exemplary microencapsulation device is described, for example, in U.S. Pat. No. 7,482,152, incorporated herein by reference in its entirety. Microfluidic droplets or emulsions can be generated by flow of a fluid to be encapsulated with an immiscible carrier fluid. For example, an oil fluid to be encapsulated can be flowed with an aqueous carrier fluid, or an aqueous fluid to be encapsulated can be flowed with an oil carrier fluid. Air can also be used as a fluid. Microfluidic droplet generators useful for microencapsulation include those employing co-flowing streams, cross-flowing streams (e.g., flow of streams at a T-junction), flow focusing, flow through perforated plates, and flow through nozzles. Droplet size can be controlled by parameters including device geometry, relative flow rates of the fluid streams, and operating pressure.

Microencapsulation can be performed at a range of operating parameters, such as different flow rates or pressures. Microencapsulation can be conducted at a pressure of at least about 10 pounds per square inch (psi), 20 psi, 30 psi, 40 psi, 50 psi, 60 psi, 70 psi, 80 psi, 90 psi, 100 psi, 200 psi, 300 psi, 400 psi, 500 psi, 600 psi, 700 psi, 800 psi, 900 psi, 1000 psi, 2000 psi, 3000 psi, 4000 psi, 5000 psi, 6000 psi, 7000 psi, 8000 psi, 9000 psi, 10000 psi, 15000 psi, 20000 psi, 25000 psi, 30000 psi, 35000 psi, 40000 psi, 45000 psi, 50000 psi, or more. Microencapsulation can be conducted at a pressure of at most about 50000 psi, 45000 psi, 40000 psi, 35000 psi, 30000 psi, 25000 psi, 20000 psi, 15000 psi, 10000 psi, 9000 psi, 8000 psi, 7000 psi, 6000 psi, 5000 psi, 4000 psi, 3000 psi, 2000 psi, 1000 psi, 900 psi, 800 psi, 700 psi, 600 psi, 500 psi, 400 psi, 300 psi, 200 psi, 100 psi, 90 psi, 80 psi, 70 psi, 60 psi, 50 psi, 40 psi, 30 psi, 20 psi, 10 psi, or less. Microencapsulation can be conducted at a pressure of about 10 pounds per square inch (psi), 20 psi, 30 psi, 40 psi, 50 psi, 60 psi, 70 psi, 80 psi, 90 psi, 100 psi, 200 psi, 300 psi, 400 psi, 500 psi, 600 psi, 700 psi, 800 psi, 900 psi, 1000 psi, 2000 psi, 3000 psi, 4000 psi, 5000 psi, 6000 psi, 7000 psi, 8000 psi, 9000 psi, 10000 psi, 15000 psi, 20000 psi, 25000 psi, 30000 psi, 35000 psi, 40000 psi, 45000 psi, or 50000 psi.

Microencapsulation can be conducted at a flow rate of at least about 1 milliliter per minute (mL/min), 2 mL/min 3 mL/min, 4 mL/min, 5 mL/min, 6 mL/min, 7 mL/min, 8 mL/min, 9 mL/min, 10 mL/min, 20 mL/min, 30 mL/min, 40 mL/min, 50 m/min, 60 mL/min, 70 mL/min, 80 mL/min, 90 mL/min, 100 mL/min, 110 mL/min, 120 m/min, 130 mL/min, 140 mL/min, 150 mL/min, 160 mL/min, 170 mL/min, 180 mL/min, 190 mL/min, 200 mL/min, 210 mL/min, 220 mL/min, 230 mL/min, 240 mL/min, 250 mL/min, 260 mL/min, 270 mL/min, 280 mL/min, 290 mL/min, 300 mL/min, 310 mL/min, 320 mL/min, 330 mL/min, 340 mL/min, 350 mL/min, 360 mL/min, 370 mL/min, 380 mL/min, 390 mL/min, 400 mL/min, 410 mL/min, 420 mL/min, 430 mL/min, 440 mL/min, 450 mL/min, 460 mL/min, 470 mL/min, 480 mL/min, 490 mL/min, 500 mL/min, or more. Microencapsulation can be conducted at a flow rate of at most about 500 mL/min, 490 mL/min, 480 mL/min, 470 mL/min, 460 mL/min, 450 mL/min, 440 mL/min, 430 mL/min, 420 mL/min, 410 mL/min, 400 mL/min, 390 mL/min, 380 mL/min, 370 mL/min, 360 mL/min, 350 mL/min, 340 mL/min, 330 mL/min, 320 mL/min, 310 mL/min, 300 mL/min, 290 mL/min, 280 mL/min, 270 mL/min, 260 mL/min, 250 mL/min, 240 mL/min, 230 mL/min, 220 mL/min, 210 mL/min, 200 mL/min, 190 mL/min, 180 mL/min, 170 mL/min, 160 mL/min, 150 mL/min, 140 mL/min, 130 mL/min, 120 mL/min, 110 mL/min, 100 m/min, 90 m/min, 80 mL/min, 70 mL/min, 60 mL/min, 50 mL/min, 40 mL/min, 30 mL/min, 20 mL/min, 10 mL/min, 9 mL/min, 8 mL/min, 7 mL/min, 6 mL/min, 5 mL/min, 4 mL/min, 3 mL/min, 2 mL/min, 1 mL/min, or less. Microencapsulation can be conducted at a flow rate of about 1 milliliter per minute (mL/min), 2 mL/min 3 mL/min, 4 mL/min, 5 mL/min, 6 mL/min, 7 mL/min, 8 mL/min, 9 mL/min, 10 mL/min, 20 mL/min, 30 mL/min, 40 mL/min, 50 m/min, 60 mL/min, 70 mL/min, 80 mL/min, 90 mL/min, 100 mL/min, 110 mL/min, 120 m/min, 130 mL/min, 140 mL/min, 150 mL/min, 160 mL/min, 170 mL/min, 180 mL/min, 190 mL/min, 200 mL/min, 210 mL/min, 220 mL/min, 230 mL/min, 240 mL/min, 250 mL/min, 260 mL/min, 270 mL/min, 280 mL/min, 290 mL/min, 300 mL/min, 310 mL/min, 320 mL/min, 330 mL/min, 340 mL/min, 350 mL/min, 360 mL/min, 370 mL/min, 380 mL/min, 390 mL/min, 400 mL/min, 410 mL/min, 420 mL/min, 430 mL/min, 440 mL/min, 450 mL/min, 460 mL/min, 470 mL/min, 480 m/min, 490 mL/min, or 500 mL/min.

Droplet generators can employ multiple parallel droplet generation operations in parallel. For example, a droplet generator (e.g., a plate, a device with channels) can employ at least 1, 2, 3, 4, 5, 6, 7, 8, 9, 10, 15, 20, 25, 30, 35, 40, 45, 50, 55, 60, 65, 70, 75, 80, 85, 90, 95, 100, or more droplet generating features (e.g., holes, channels, nozzles). A droplet generator can employ at most 100, 95, 90, 85, 80, 75, 70, 65, 60, 55, 50, 45, 40, 35, 30, 25, 20, 15, 10, 9, 8, 7, 6, 5, 4, 3, 2, or 1 droplet generating features. A droplet generator can employ about 1, 2, 3, 4, 5, 6, 7, 8, 9, 10, 15, 20, 25, 30, 35, 40, 45, 50, 55, 60, 65, 70, 75, 80, 85, 90, 95, 100, or more droplet generating features.

Microencapsulation can be performed via an emulsification process. For example, cannabinoid compositions can be emulsified in a mixer, such as an agitator, impeller, centrifugal mixer, or high-shear mixer. High-shear mixers can include batch high-shear mixers and inline high-shear mixers (e.g., rotor-stator mixers). Emulsification can also be conducted without a mixer, by combining fluids thermodynamically favored to form an emulsion, optionally with the aid of one or more emulsifiers or surfactants.

Microencapsulation processes can be conducted with the aid of one or more emulsifiers or surfactants. Emulsifiers and surfactants can include but are not limited to saponins (e.g., quillaja tree extract such as Q-NATURALE®, yucca extract), lecithin, soy lecithin, mustard seed hull extract, sodium stearoyl lactylate, polysorbate 20, and combinations thereof.

Microcapsules can comprise one or more stabilizers or gelling agents, which can be used to stabilize a microcapsule or emulsion. Stabilizers or gelling agents can include but are not limited to alginate (also algin or alginic acid) and agar. Alginate can be used in a variety of forms, including but not limited to inorganic salts such as sodium alginate, potassium alginate, calcium alginate, and combinations thereof. Alginate can be derived from sources such as seaweed (e.g., Macrocystis pyrifera, Ascophyllum nodosum, Laminaria spp.) or bacteria (e.g., Pseudomonas spp., Azotobacter spp.). Cross-linking agents or solutions, such as calcium chloride, can be used to stabilize or gel microcapsules.

Microcapsules can be characterized by a size (e.g., a diameter). The microcapsule size can be about 0.154 micrometers. The microcapsule size can be less than or equal to about 0.154 micrometers. The microcapsule size can be greater than or equal to about 0.154 micrometers. The microcapsule size can be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 micrometers. The microcapsule size can be less than or equal to about 500, 450, 400, 350, 300, 250, 200, 150, 100, 95, 90, 85, 80, 75, 70, 65, 60, 55, 50, 45, 40, 35, 30, 25, 20, 15, 10, 9.5, 9, 8.5, 8, 7.5, 7, 6.5, 6, 5.5, 5, 4.5, 4, 3.5, 3, 2.5, 2, 1.5, 1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001 micrometers, or less. The microcapsule size can be greater than or equal to about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 micrometers. The microcapsule size can be from about 0.1 to about 0.2 micrometers. The microcapsule size can be from about 0.05 to about 0.25 micrometers. The microcapsule size can be from about 0.05 to about 0.55 micrometers. The microcapsule size can be from about 0.05 to about 1 micrometer. The size distribution in a population of microcapsules can be homogeneous or substantially homogeneous. For example, a population of microcapsules can be characterized by dispersity, or polydispersity index (PDI), of less than or equal to about 20, 19, 18, 17, 16, 16, 15, 14, 13, 12, 11, 10, 9, 8, 7, 6, 5, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.45, 1.40, 1.35, 1.30, 1.25, 1.20, 1.15, 1.14, 1.13, 1.12, 1.11, 1.10, 1.09, 1.08, 1.07, 1.06, 1.05, 1.04, 1.03, 1.02, 1.01, or 1.00.

Encapsulated cannabinoids can be present in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50 micrograms, or more per microcapsule. Encapsulated cannabinoids can be present in a quantity of at most about 50, 45, 40, 35, 30, 25, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, 0.2, 0.1 micrograms, or less per microcapsule. Encapsulated cannabinoids can be present in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated cannabinoids can be present in a quantity of from about 1 to about 10 micrograms per microcapsule. Encapsulated cannabinoids can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or more by weight of a microcapsule. Encapsulated cannabinoids can be present in a quantity of at most about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.10%, or less by weight of a microcapsule. Encapsulated cannabinoids can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule.

Cannabinoids can be present in a product, such as a food product, in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500 milligrams (mg), or more. Cannabinoids can be present in a product, such as a food product, in a quantity of at most about 500, 450, 400, 350, 300, 250, 200, 150, 100, 90, 80, 70, 60, 50, 40, 30, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, 0.2, 0.1 mg, or less. Cannabinoids can be present in a product, such as a food product, in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 mg. Cannabinoids can be present in a product, such as a food product, in a quantity of from about 50 to about 150 milligrams. Cannabinoids can be present in a product, such as a food product, in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or more by weight of the product. Cannabinoids can be present in a product, such as a food product, in a quantity of at most about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, or less by weight of the product. Cannabinoids can be present in a product, such as a food product, in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

The cannabinoids of the compositions disclosed herein can comprise cannabidiol-class compounds, including but not limited to cannabidiol (CBD), cannabidiolic acid (CBDA), cannabidiol monomethylether (CBDM), cannabidiol-C₄ (CBD-C₄), cannabidivarin (CBDV), cannabidivarinic acid (CBDVA), cannabidiorcol (CBD-C₁), and combinations thereof. CBD can comprise delta-1-cannabidiol, delta-2-cannabidiol, delta-3-cannabidiol, delta-3,7-cannabidiol, delta-4-cannabidiol, delta-5-cannabidiol, delta-6-cannabidiol, and combinations thereof.

Encapsulated cannabidiol compounds can be present in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50 micrograms, or more per microcapsule. Encapsulated cannabidiol compounds can be present in a quantity of at most about 50, 45, 40, 35, 30, 25, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, 0.2, 0.1 micrograms, or less per microcapsule. Encapsulated cannabidiol compounds can be present in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated cannabidiol compounds can be present in a quantity of from about 1 to about 10 micrograms per microcapsule. Encapsulated cannabidiol compounds can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or more by weight of a microcapsule. Encapsulated cannabidiol compounds can be present in a quantity of at most about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or less by weight of a microcapsule. Encapsulated cannabidiol compounds can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule.

Cannabidiol compounds can be present in a product, such as a food product, in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500 milligrams (mg), or more. Cannabidiol compounds can be present in a product, such as a food product, in a quantity of at most about 50, 45, 40, 35, 30, 25, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, 0.2, 0.1 milligrams (mg), or less. Cannabidiol compounds can be present in a product, such as a food product, in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabidiol compounds can be present in a product, such as a food product, in a quantity of from about 50 to about 150 milligrams. Cannabidiol compounds can be present in a product, such as a food product, in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20, 25%, 30%, 35%, 40%, 45%, 50%, or more by weight of the product. Cannabidiol compounds can be present in a product, such as a food product, in a quantity of at most about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or less by weight of the product. Cannabidiol compounds can be present in a product, such as a food product, in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

The compositions of the present disclosure can comprise tetrahydrocannabinol (THC) as a type of cannabinoid. THC can comprise delta-9-THC, delta-8-THC, and combinations thereof. THC can comprise delta-6a,7-tetrahydrocannabinol, delta-7-tetrahydrocannabinol, delta-8-tetrahydrocannabinol, delta-9,11-tetrahydrocannabinol, delta-9-tetrahydrocannabinol, delta-10-tetrahydrocannabinol, delta-6a,10a-tetrahydrocannabinol, and combinations thereof. Delta-9-tetrahydrocannabinol can comprise stereoisomers including (6aR,10aR)-delta-9-tetrahydrocannabinol, (6aS,10aR)-delta-9-tetrahydrocannabinol, (6aS,10aS)-delta-9-tetrahydrocannabinol, (6aR,10aS)-delta-9-tetrahydrocannabinol, and combinations thereof.

In cases where the cannabinoid compositions comprise microcapsules, THC compounds can be present in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50 micrograms, or more per microcapsule. Encapsulated THC compounds can be present in a quantity of at most about 50, 45, 40, 35, 30, 25, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, 0.2, 0.1 micrograms, or less per microcapsule. Encapsulated THC compounds can be present in a quantity of from about 1 to about 10 micrograms per microcapsule. Encapsulated THC compounds can be present in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated THC compounds can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or more by weight of a microcapsule. Encapsulated THC compounds can be present in a quantity of at most about 50%, 45%, 40%, 35%, 30%, 25%, 20%, 19%, 18%, 17%, 16%, 1%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or less by weight of a microcapsule. Encapsulated THC compounds can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 1%, 16%, 17%, 18%, 19%, 20%, 25%, 30%, 35%, 40%, 45%, or 50% by weight of a microcapsule.

THC compounds can be present in a cannabinoid composition in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500 mg, or more. THC compounds can be present in a cannabinoid composition in a quantity of at most about 500, 450, 400, 350, 300, 250, 200, 150, 100, 90, 80, 70, 60, 50, 40, 30, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, 0.2, 0.1 mg, or less. THC compounds can be present in a cannabinoid composition in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 mg. THC compounds can be present in a cannabinoid composition in a quantity of from about 50 to about 150 milligrams. THC compounds can be present in a cannabinoid composition in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%1, 3%, 14%, 15%, 16%, 17%, 18%, 19%, 20%, 25%, 30%, 35%, 40%, 45%, 50%, or more by weight of the product. THC compounds can be present in cannabinoid composition in a quantity of at most about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, or less by weight of the product. THC compounds can be present in a cannabinoid composition in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

In some cases, a composition of the present disclosure does not contain a psychoactive amount of THC. For example, cannabinoids in compositions of the present disclosure can contain less than about 100%, 90%, 80%, 70%, 60%, 50%, 40%, 30%, 20%, 10%, 5%, 1%, 0.7%, 0.5%, 0.3%, or 0.1% THC relative to the total quantity of cannabinoid compounds. In some cases, the ratio of a non-THC cannabinoid (e.g., cannabidiol) to THC in a composition of the present disclosure is greater than or equal to about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, or 100:1 (non-THC cannabinoid:THC). In some cases, compositions of the present disclosure contain less than about 0.3% THC by weight.

The cannabinoid compositions of the present disclosure can comprise one or more terpene compounds, including but not limited to terpenoids such as monoterpenoids, sesquiterpenoids, diterpenoids, and triterpenoids. Terpenes can be acyclic, monocyclic, or polycyclic. Terpenes can include but are not limited to myrcene, limonene, linalool, trans-ocimene, cis-ocimene, alpha-pinene, beta-pinene, alpha-humulene (alpha-caryophyllene), beta-caryophyllene, delta-3-carene, trans-gamma-bisabolene, cis-gamma-bisabolene, trans-alpha-famesene, cis-beta-farnesene, beta-fenchol, beta-phellandrene, guajol, alpha-gualene, alpha-eudesmol, beta-eudesmol, gamma-eudesmol, terpinolene, alpha-selinene, beta-selinene, alpha-terpineol, fenchone, camphene, cis-sabinene hydrate, alpha-trans-bergamotene, alpha-cis-bergamotene, borneol, gamma-curcumene, alpha-thujene, epi-alpha-bisabolol, ipsdienol, alpha-ylangene, beta-elemene, gamma-muurolene, alpha-cadinene, alpha-longipinene, caryophyllene oxide, and combinations thereof.

Encapsulated terpenes can be present in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50 micrograms, or more per microcapsule. Encapsulated terpenes can be present in a quantity of at most about 50, 45, 40, 35, 30, 25, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, 0.2, 0.1 micrograms, or less per microcapsule. Encapsulated terpenes can be present in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated terpene compounds can be present in a quantity of from about 1 to about 10 micrograms per microcapsule. Encapsulated terpenes can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, %, 12%, 13%, 14%, 1%, 16%, 17, 18%, %19%, 20%, 25%, 30%, 35%, 40%, 45%, 50%, or more by weight of a microcapsule. Encapsulated terpenes can be present in a quantity of at most about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or less by weight of a microcapsule. Encapsulated terpenes can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 1%, 2%, %13%, %14%, 15%, 16%, 17%, 18%, 19%, 20%, 25%, 30%, 35%, 40%, 45%, or 50% by weight of a microcapsule.

Terpene compounds can be present in a product, such as a food product, in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500 mg, or more. Terpene compounds can be present in a product, such as a food product, in a quantity of at most about 500, 450, 400, 350, 300, 250, 200, 150, 100, 90, 80, 70, 60, 50, 40, 30, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, 0.2, 0.1 mg, or less. Terpene compounds can be present in a product, such as a food product, in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 mg. Terpene compounds can be present in a cannabinoid composition in a quantity of from about 50 to about 150 milligrams. Terpene compounds can be present in a cannabinoid composition in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or more by weight of the product. Terpene compounds can be present in a cannabinoid composition in a quantity of at most about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, or less by weight of the product. Terpene compounds can be present in a cannabinoid composition in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

The cannabinoid compositions of the present disclosure can be enriched in cannabinoids compared to hemp oil. For example, a cannabinoid composition can comprise hemp oil and cannabinoids from plant sources such as extracts (e.g., hemp extract) and essential oils. A composition can comprise about 0%, 10%, 20%, 30%, 40%, 50%, 60%, 70%, 80%, 90%, 100%, 200%, 300%, 400%, 500%, 600%, 700%, 800%, 900%, 1000%, or greater concentration of cannabinoids compared to hemp oil.

The cannabinoid compositions of the present disclosure can be enriched in cannabidiol compounds compared to hemp oil. For example, a cannabinoid composition can comprise hemp oil and cannabidiol compounds from plant sources such as extracts (e.g., hemp extract) and essential oils. A composition can comprise about 0%, 10%, 20%, 30%, 40%, 50%, 60%, 70%, 80%, 90%, 100%, 200%, 300%, 400%, 500%, 600%, 700%, 800%, 900%, 1000%, or greater concentration of cannabidiol compounds compared to hemp oil.

The cannabinoid compositions of the present disclosure can be enriched in THC compounds compared to hemp oil. For example, a cannabinoid composition can comprise hemp oil and THC compounds from plant sources such as extracts (e.g., hemp extract) and essential oils. A cannabinoid composition can comprise about 0%, 10%, 20%, 30%, 40%, 50%, 60%, 70%, 80%, 90%, 100%, 200%, 300%, 400%, 500%, 600%, 700%, 800%, 900%, 1000%, or greater concentration of THC compounds compared to hemp oil.

The cannabinoid compositions of the present disclosure can be enriched in terpenes compared to hemp oil. For example, a cannabinoid composition can comprise hemp oil and terpenes from plant sources such as extracts (e.g., hemp extract) and essential oils. A cannabinoid composition can comprise about 0%, 10%, 20%, 30%, 40%, 50%, 60%, 70%, 80%, 90%, 100%, 200%, 300%, 400%, 500%, 600%, 700%, 800%, 900%, 1000%, or greater concentration of terpenes compared to hemp oil.

Cannabinoid compositions of the present disclosure may be used to treat various diseases or conditions in subjects (e.g., humans, mammals, vertebrates), including but not limited to ALS, Alzheimer's, antibacterial resistant infections, anxiety, atherosclerosis, arthritis, asthma, cancer, colitis, Crohn's, diabetes, depression, endocrine disorders, epilepsy, seizures, fibromyalgia, glaucoma, heart disease, Huntington's, inflammation, irritable bowel syndrome (IBS), kidney disease, liver disease, motion sickness, nausea, neurodegeneration, neuropathic pain, neuropathy, obesity, obsessive compulsive disorder (OCD), osteoporosis, Parkinson's, prion diseases, Mad Cow disease, post-traumatic stress disorder (PTSD), rheumatism, schizophrenia, sickle cell anemia, skin conditions (e.g., psoriasis, dermatitis, allergic inflammation, chronic pruritus), sleep disorders (e.g., sleep-wake disorders, apnea), spinal cord injury, stress, stroke, and traumatic brain injury (TBI). Cannabinoid compositions of the present disclosure may be used as a preventive pharmaceutical to inhibit negative health processes (e.g. inflammation caused by oxidative stress) or promote positive health processes (e.g. microbial inhibition).

Methods for Optimizing Cannabinoid Dosage

The optimal or target dosage of a cannabinoid composition may vary depending upon the individual, a point in time, the chemical composition of the composition, and/or the intended application of the composition. The target dosage of a cannabinoid composition may be tied to genetic and environmental factors. The target dosage of a cannabinoid composition may vary between separate individuals. The target dosage of a cannabinoid composition may vary with time for a particular individual. The target dosage may be determined using measurable biomarkers. Measurable biomarkers may include, for example, age, height, weight, resting heart rate, blood oxygen level, systolic blood pressure, diastolic blood pressure, basal body temperature, blood sugar, and oxidative stress level, among other factors, and/or a combination thereof. Measurable biomarkers may be measured for a given individual at an instance or over time to assist in determining, confirming, and/or estimating a target dosage level of a cannabinoid composition for the given individual. In some cases, a target dosage level of a cannabinoid composition for a given individual may be an ideal dosage level of the cannabinoid composition for the given individual, e.g., for a particular day or for a plurality of days.

In some instances, one or more biomarkers of a given individual may be measured after application of a volume of a cannabinoid composition, and the data from such post-application measurement may be used to determine whether the volume of the cannabinoid composition is indicative of an optimal dosage for the given individual. In some instances, one or more biomarkers of a given individual may be measured prior to, and subsequent to application of a volume of a cannabinoid composition, and the data from such pre-application and post-application, and any changes therebetween, may be used to determine whether the volume of the cannabinoid composition is indicative of an optimal dosage for the given individual. In some instances, one or more biomarkers of a given individual may be measured prior to, during, and/or subsequent to application of a volume of a cannabinoid composition over a period of time, with multiple measurements during the period of time, and the data used to determine whether the volume of the cannabinoid composition is indicative of an optimal dosage for the given individual. In some instances, a measurable personal biomarker may be brought closer to a target value for the personal biomarker by the consumption of one or more optimal doses of a cannabinoid composition. For example, an individual with high blood pressure may see a reduction toward a normal blood pressure by the application of a target dosage of a cannabinoid composition. Any of the above measurement protocols may be repeated with application of different volumes of the cannabinoid composition to determine whether one or more volumes of the cannabinoid composition is indicative of an optimal dosage for the given individual. In some instances, the multiple volumes may be applied at regular time intervals. Alternatively, the multiple volumes may be applied at irregular time intervals. In some instances, the multiple volumes may be applied at a particular state of a biomarker measurement (e.g., when the heart rate is at a resting heart rate for the given individual). In some instances, the time interval between one or more data points may be used to determine whether a volume of the cannabinoid composition is indicative of an optimal dosage for the given individual, such as the duration of time it takes for a measurable biomarker to meet a condition (e.g., to reach a predetermined threshold value from an initial value, such as an average value), the duration of time it takes for a measurable biomarker to change by a predetermined value from an initial value (e.g., average value), the duration of time it takes for a measurable biomarker to reach a maximum value (e.g., peak) from an initial value (e.g., average value), the duration of time it takes for a measurable biomarker to return from a maximum value to an initial value (e.g., average value), the duration of time it takes for a measurable biomarker to return to an initial value (e.g., average value) after application of the volume of the cannabinoid composition, and the like.

The target dosage of a cannabinoid composition may be determined by the measurement of one or more biological signals (or biological markers as used interchangeably herein) of an individual. The one or more biological signals of the individual may comprise: a heart rate (e.g., a resting heart rate), a breathing rate, an oxygen level (e.g., a blood oxygen level), blood pressure (e.g., systolic blood pressure, diastolic blood pressure), a temperature (e.g., a basal body temperature), a perspiration, a concentration of an analyte (e.g., sugar/glucose, antibody) in a bodily fluid (e.g., blood, urine), pH of a bodily fluid, an invariant biomarker, a level of oxidative stress, and/or an amount of an analyte (e.g., reactive oxygen species) in a cell of the individual.

The one or more biological signals of the individual may be measured by one or more sensors (e.g., biosensors). The one or more sensors may comprise an electrochemical sensor, an optical sensor, an electronic sensor, a piezoelectric sensor, a gravimetric sensor, a pyroelectric sensor, etc. Examples of the one or more sensors can include, but are not limited to, a glucose sensor, heart rate monitor, galvanic skin response (GSR) sensor, skin temperature sensor, capacitive sensor, metabolic sensor, blood pressure monitors, sweat sensors, electrocardiogram (ECG) monitors, bioelectrical impedance analysis (BIA) monitor (e.g., a body fat monitor), antibody/antigen interaction sensor, etc.

In some cases, an initial value of a biological signal of an individual may be detected (e.g., using a biosensor) prior to at least one application of a cannabinoid composition to the individual (e.g., to a region of a mouth of the individual). Upon the at least one application of the cannabinoid compound, an additional value of the biological signal of the individual may be detected.

In some cases, an initial value of a biological signal of an individual may be detected (e.g., using a biosensor) prior to at least one application of a cannabinoid composition to the individual (e.g., to a region of a mouth of the individual). Following, a cannabinoid composition may be administered to the individual (e.g., to a region of a mouth of the individual), and a first taste for the cannabinoid composition may be determined. Subsequently, at least one additional cannabinoid composition may be administered to the individual (e.g., to a region of a mouth of the individual), and a second taste for the cannabinoid composition may be determined. In such cases, an additional value of the biological signal of the individual may be detected upon detecting a change in characterization of the first taste and the second taste. Optionally, such process may be repeated until a change between the initial value and the additional value of the biological signal is detected, e.g., closer to a predetermined target value. A cumulative volume of the applied cannabinoid composition may be determined to be a target dosage of the cannabinoid composition for the individual when the change in the value of the biological signal is detected.

A target dosage of the cannabinoid composition may be determined when the additional value of the biological signal meets a condition. The condition may be a predetermined condition. The condition may be met, for example, when: (i) the additional value of the biological signal is closer to a target value of the biological signal as compared to the initial value of the biological signal, (ii) the additional value of the biological signal is further away from a target value of the biological signal as compared to the initial value of the biological signal, (iii) the additional value of the biological signal is within a predetermined range around a target value of the biological signal, and the initial value is not within the predetermined range, (iv) the additional value of the biological signal is within a predetermined range around a target value of the biological signal, and the initial value is within the predetermined range, or (v) a change between the initial value and the additional value of the biological signal is greater than a predetermined level (e.g., greater than 10%).

The condition may be met when the additional value of the biological signal is closer to a target value of the biological signal as compared to the initial value of the biological signal. The additional value may be closer to the target value of the biological signal by at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, or more, as compared to the initial value of the biological signal. The additional value may be closer to the target value of the biological signal by at most about 500%, 400%, 300%, 200%, 100%, 90%, 80%, 70%, 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or less, as compared to the initial value of the biological signal.

The condition may be met when the additional value of the biological signal is further away from a target value of the biological signal as compared to the initial value of the biological signal. The additional value may be further away from the target value of the biological signal by at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%4, 40%, 50%, 60%, 70%, 80%, 90%, 100%, 200%, 300%, 400%, 500%, or more, as compared to the initial value of the biological signal. The additional value may be further away from the target value of the biological signal by at most about 500%, 400%, 300%, 200%, 100%, 90%, 80%, 70%, 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or less, as compared to the initial value of the biological signal.

The condition may be met when the additional value of the biological signal is within a predetermined range around a target value of the biological signal, and the initial value is not within the predetermined range. The predetermined range may be within at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or more of the target value. The predetermined range may be within at most about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or less of the target value. The predetermined range may include values greater than or equal to the target value. The predetermined range may include values less than or equal to the target value.

The condition may be met when a change between the initial value and the additional value of the biological signal is greater than a predetermined level. The change may be such that the additional value is greater than the initial value. Alternatively, the change may be such that the additional value is less than the initial value. The predetermined level may be at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, or more relative to the initial value. The predetermined value may be at most about 500%, 400%, 300%, 200%, 100%, 90%, 80%, 70%, 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or less relative to the initial value.

The additional value of the biological signal of the individual may be detected at least about 1 min, 5 mins, 10 mins, 30 mins, 60 mins, 2 hours, 4 hours, 6 hours, 12 hours, 24 hours, or more following the at least one application of the cannabinoid compound to the individual. The additional value of the biological signal of the individual may be detected at most about 24 hours, 12 hours, 6 hours, 4 hours, 2 hours, 60 mins, 30 mins, 10 mins, 5 mins, 1 min, or less following the at least one application of the cannabinoid compound to the individual.

At least 1, 2, 3, 4, 5, or more biological signals of the individual may be analyzed to determine the target dosage of the cannabinoid composition. At most 5, 4, 3, 2, or 1 biological signal of the individual may be analyzed to determine the target dosage of the cannabinoid composition.

The target dosage of a cannabinoid composition may be determined by the measurement of a resting heart rate. A resting heart rate may be defined as the average heart rate of an individual who is not exerting or otherwise in a state of high cardiac output. A resting heart rate may be measured by a device such as a pulse monitor or a wearable device (e.g. a fitness watch). A resting heart rate may be measured in good health, during a chronic disease state, or during an infective disease state. A resting heart rate may be measured over a defined period of time, e.g. 30 seconds or 1 minute. A resting heart rate may be continuously monitored during the administration of a cannabinoid composition. In some instances, a resting heart rate may be measured before the administration of a cannabinoid composition. A resting heart rate may be subsequently measured after the administration of a cannabinoid composition to determine an effect of the cannabinoid composition on resting heart rate. In other instances, a resting heart rate may be measured only after the administration of a cannabinoid composition. In some instances, a resting heart rate may be repeatedly measured between multiple administrations of a cannabinoid composition until a target dosage or a target resting heart rate is achieved. In some instances, the target dosage of a given cannabinoid composition may be determined when a resting heart rate is about 30 beats per minute (bpm), 35 bpm, 40 bpm, 45 bpm, 50 bpm, 51 bpm, 52 bpm, 53 bpm, 54 bpm, 55 bpm, 56 bpm, 57 bpm, 58 bpm, 59 bpm, 60 bpm, 61 bpm, 62 bpm, 63 bpm, 64 bpm, 65 bpm, 66 bpm, 67 bpm, 68 bpm, 69 bpm, 70 bpm, 75 bpm, 80 bpm, 85 bpm, 90 bpm, 95 bpm, 100 bpm, 105 bpm, 110 bpm, 115 bpm, or about 120 bpm. The target dosage of a given cannabinoid composition may be determined when a resting heart rate is at least about 30 bpm, 35 bpm, 40 bpm, 45 bpm, 50 bpm, 51 bpm, 52 bpm, 53 bpm, 54 bpm, 55 bpm, 56 bpm, 57 bpm, 58 bpm, 59 bpm, 60 bpm, 61 bpm, 62 bpm, 63 bpm, 64 bpm, 65 bpm, 66 bpm, 67 bpm, 68 bpm, 69 bpm, 70 bpm, 75 bpm, 80 bpm, 85 bpm, 90 bpm, 95 bpm, 100 bpm, 105 bpm, 110 bpm, 115 bpm, 120 bpm, or more. The target dosage of a given cannabinoid composition may be determined when a resting heart rate is at most about 120 bpm, 115 bpm, 110 bpm, 105 bpm, 100 bpm, 95 bpm, 90 bpm, 85 bpm, 80 bpm, 75 bpm, 70 bpm, 69 bpm, 68 bpm, 67 bpm, 66 bpm, 65 bpm, 64 bpm, 63 bpm, 62 bpm, 61 bpm, 60 bpm, 59 bpm, 58 bpm, 57 bpm, 56 bpm, 55 bpm, 54 bpm, 53 bpm, 52 bpm, 51 bpm, 50 bpm, 45 bpm, 40 bpm, 35 bpm, 30 bpm, or less. A target dosage for a given cannabinoid composition may be determined when a resting heart rate is in a range from about 30 bpm to about 40 bpm, about 30 bpm to about 50 bpm, about 30 bpm to about 60 bpm, about 30 bpm to about 70 bpm, about 30 bpm to about 80 bpm, about 30 bpm to about 90 bpm, about 30 bpm to about 100 bpm, about 30 bpm to about 110 bpm, about 30 bpm to about 120 bpm, about 40 bpm to about 50 bpm, about 40 bpm to about 60 bpm, about 40 bpm to about 70 bpm, about 40 bpm to about 80 bpm, about 40 bpm to about 90 bpm, about 40 bpm to about 100 bpm, about 40 bpm to about 110 bpm, about 40 bpm to about 120 bpm, about 50 bpm to about 60 bpm, about 50 bpm to about 70 bpm, about 50 bpm to about 80 bpm, about 50 bpm to about 90 bpm, about 50 bpm to about 100 bpm, about 50 bpm to about 110 bpm, about 50 bpm to about 120 bpm, about 60 bpm to about 70 bpm, about 60 bpm to about 80 bpm, about 60 bpm to about 90 bpm, about 60 bpm to about 100 bpm, about 60 bpm to about 110 bpm, about 60 bpm to about 120 bpm, about 70 bpm to about 80 bpm, about 70 bpm to about 90 bpm, about 70 bpm to about 100 bpm, about 70 bpm to about 110 bpm, about 70 bpm to about 120 bpm, about 80 bpm to about 90 bpm, about 80 bpm to about 100 bpm, about 80 bpm to about 110 bpm, about 80 bpm to about 120 bpm, about 90 bpm to about 100 bpm, about 90 bpm to about 110 bpm, about 90 bpm to about 120 bpm, about 100 bpm to about 110 bpm, about 100 bpm to about 120 bpm, or about 110 bpm to about 120 bpm.

The target dosage of a cannabinoid composition may be determined by the measurement of a blood oxygen level. A blood oxygen level may be determined by a sensor or other device, e.g. a pulse-ox sensor. A blood oxygen level may be measured in good health, during a chronic disease state, or during an infective disease state. A blood oxygen level may be measured over a defined period of time, e.g. 30 seconds or 1 minute. In some instances, a blood oxygen level may be continuously monitored throughout the administration of a cannabinoid composition. In some instances, a blood oxygen level may be measured before the administration of a cannabinoid composition. A blood oxygen level may be subsequently measured after the administration of a cannabinoid composition to determine an effect of the cannabinoid composition on blood oxygen level. In other instances, a blood oxygen level may be measured only after the administration of a cannabinoid composition. In some instances, a blood oxygen level may be repeatedly measured between multiple administrations of a cannabinoid composition until a target dosage or a target blood oxygen level is achieved. The target dosage of a given cannabinoid composition may be determined when a blood oxygen level is about 80%, 81%, 82%, 83%, 84%, 85%, 86%, 87%, 88%, 89%, 90%, 91%, 92%, 93%, 94%, 95%, 96%, 97%, 98%, 99%, or about 100%. The target dosage of a given cannabinoid composition may be determined when a blood oxygen level is at least about 80%, 81%, 82%, 83%, 84%, 85%, 86%, 87%, 88%, 89%, 90%, 91%, 92%, 93%, 94%, 95%, 96%, 97%, 98%, 99%, or at least about 100%. The target dosage of a given cannabinoid composition may be determined when a blood oxygen level is at most about 100%, 99%, 98%, 97%, 96%, 95%, 94%, 93%, 92%, 91%, 90%, 89%, 88%, 87%, 86%, 85%, 84%, 83%, 82%, 81%, 80%, or less. A target dosage of a given cannabinoid composition may be determined for a blood oxygen level in a range from about 80% to about 85%, about 80% to about 90%, about 80% to about 95%, about 80% to about 96%, about 80% to about 97%, about 80% to about 98%, about 80% to about 99%, about 80% to about 100%, about 85% to about 90%, about 85% to about 95%, about 85% to about 96%, about 85% to about 97%, about 85% to about 98%, about 85% to about 99%, about 85% to about 100%, about 90% to about 95%, about 90% to about 96%, about 90% to about 97%, about 90% to about 98%, about 90% to about 99%, about 90% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%.

The target dosage of a cannabinoid composition may be determined by the measurement of a systolic blood pressure. A systolic blood pressure may be determined by a sensor or other device, e.g. a blood pressure cuff. A systolic blood pressure may be measured in good health, during a chronic disease state, or during an infective disease state. A systolic blood pressure may be measured over a defined period of time, e.g. 30 seconds or 1 minute. In some instances, a systolic blood pressure may be continuously monitored throughout the administration of a cannabinoid composition. In some instances, a systolic blood pressure may be measured before the administration of a cannabinoid composition. A systolic blood pressure may be subsequently measured after the administration of a cannabinoid composition to determine an effect of the cannabinoid composition on systolic blood pressure. In other instances, a systolic blood pressure may be measured only after the administration of a cannabinoid composition. In some instances, a systolic blood pressure may be repeatedly measured between multiple administrations of a cannabinoid composition until a target dosage or a target systolic blood pressure is achieved. The target dosage of a given cannabinoid composition may be determined when a systolic blood pressure is about 70 millimeters of mercury (mmHg), 80 mmHg, 90 mmHg, 100 mmHg, 110 mmHg, 120 mmHg, 130 mmHg, 140 mmHg, 150 mmHg, 160 mmHg, 170 mmHg, 180 mmHg, 190 mmHg, or about 200 mmHg. The target dosage of a given cannabinoid composition may be determined when a systolic blood pressure is at least about 70 mmHg, 80 mmHg, 90 mmHg, 100 mmHg, 110 mmHg, 120 mmHg, 130 mmHg, 140 mmHg, 150 mmHg, 160 mmHg, 170 mmHg, 180 mmHg, 190 mmHg, 200 mmHg, or more. The target dosage of a cannabinoid composition may be determined for a systolic blood pressure of at most about 200 mmHg, 190 mmHg, 180 mmHg, 170 mmHg, 160 mmHg, 150 mmHg, 140 mmHg, 130 mmHg, 120 mmHg, 110 mmHg, 100 mmHg, 90 mmHg, 80 mmHg, 70 mmHg, or less. The target dosage of a given cannabinoid composition may be determined for a systolic blood pressure range from about 70 mmHg to about 80 mmHg, about 70 mmHg to about 90 mmHg, about 70 mmHg to about 100 mmHg, about 70 mmHg to about 120 mmHg, about 70 mmHg to about 140 mmHg, about 70 mmHg to about 160 mmHg, about 70 mmHg to about 180 mmHg, about 70 mmHg to about 200 mmHg, about 80 mmHg to about 90 mmHg, about 80 mmHg to about 100 mmHg, about 80 mmHg to about 120 mmHg, about 80 mmHg to about 140 mmHg, about 80 mmHg to about 160 mmHg, about 80 mmHg to about 180 mmHg, about 80 mmHg to about 200 mmHg, about 100 mmHg to about 120 mmHg, about 100 mmHg to about 140 mmHg, about 100 mmHg to about 160 mmHg, about 100 mmHg to about 180 mmHg, about 100 mmHg to about 200 mmHg, about 120 mmHg to about 140 mmHg, about 120 mmHg to about 160 mmHg, about 120 mmHg to about 180 mmHg, about 120 mmHg to about 200 mmHg, about 140 mmHg to about 160 mmHg, about 140 mmHg to about 180 mmHg, about 140 mmHg to about 200 mmHg, about 160 mmHg to about 180 mmHg, about 160 mmHg to about 200 mmHg, or about 180 mmHg to about 200 mmHg.

The target dosage of a cannabinoid composition may be determined by the measurement of a diastolic blood pressure. A diastolic blood pressure may be determined by a sensor or other device, e.g. a blood pressure cuff. A diastolic blood pressure may be measured in good health, during a chronic disease state, or during an infective disease state. A diastolic blood pressure may be measured over a defined period of time, e.g. 30 seconds or 1 minute. In some instances, a diastolic blood pressure may be continuously monitored throughout the administration of a cannabinoid composition. In some instances, a diastolic blood pressure may be measured before the administration of a cannabinoid composition. A diastolic blood pressure may be subsequently measured after the administration of a cannabinoid composition to determine an effect of the cannabinoid composition on diastolic blood pressure. In other instances, a diastolic blood pressure may be measured only after the administration of a cannabinoid composition. In some instances, a diastolic blood pressure may be repeatedly measured between multiple administrations of a cannabinoid composition until a target dosage or a target diastolic blood pressure is achieved. The target dosage of a given cannabinoid composition may be determined when a diastolic blood pressure is about 40 mmHg, 50 mmHg, 60 mmHg, 70 mmHg, 80 mmHg, 90 mmHg, 100 mmHg, 110 mmHg, 120 mmHg, 130 mmHg, or about 140 mmHg. The target dosage of a given cannabinoid composition may be determined when a diastolic blood pressure is at least about 40 mmHg, 50 mmHg, 60 mmHg, 70 mmHg, 80 mmHg, 90 mmHg, 100 mmHg, 110 mmHg, 120 mmHg, 130 mmHg, 140 mmHg, or more. The target dosage of a cannabinoid composition may be determined when a diastolic blood pressure is at most about 140 mmHg, 130 mmHg, 120 mmHg, 110 mmHg, 100 mmHg, 90 mmHg, 80 mmHg, 70 mmHg, 60 mmHg, 50 mmHg, 40 mmHg, or less. The target dosage of a given cannabinoid composition may be determined for a diastolic blood pressure range from about 40 mmHg to about 60 mmHg, 40 mmHg to about 80 mmHg, 40 mmHg to about 100 mmHg, 40 mmHg to about 120 mmHg, 40 mmHg to about 140 mmHg, 60 mmHg to about 80 mmHg, 60 mmHg to about 100 mmHg, 60 mmHg to about 120 mmHg, 60 mmHg to about 140 mmHg, 80 mmHg to about 100 mmHg, 80 mmHg to about 120 mmHg, 80 mmHg to about 140 mmHg, 100 mmHg to about 120 mmHg, 100 mmHg to about 140 mmHg, or about 120 mmHg to about 140 mmHg.

The target dosage of a cannabinoid composition may be determined by the measurement of a basal body temperature. A basal body temperature may be defined as the maximum internal temperature of the body. A basal body temperature may be measured in good health, during a chronic disease state, or during an infective disease state. A basal body temperature may be measured internally (e.g. anorectal measurement) or externally (e.g. skin measurement). A basal body temperature may be determined by a sensor or other device, e.g. a thermometer. A basal body temperature may be measured over a defined period of time, e.g. 30 seconds or 1 minute. In some instances, a basal body temperature may be continuously monitored throughout the administration of a cannabinoid composition. In some instances, a basal body temperature may be measured before the administration of a cannabinoid composition. A basal body temperature may be subsequently measured after the administration of a cannabinoid composition to determine an effect of the cannabinoid composition on basal body temperature. In other instances, a basal body temperature may be measured only after the administration of a cannabinoid composition. In some instances, a basal body temperature may be repeatedly measured between multiple administrations of a cannabinoid composition until a target dosage or a target basal body temperature is achieved. The target dosage of a given cannabinoid composition may be determined when a basal body temperature is about 97.5 degrees Fahrenheit (° F.), 97.6° F., 97.7° F., 97.8° F., 97.9° F., 98.0° F., 98.1° F., 98.2° F., 98.3° F., 98.4° F., 98.5° F., 98.6° F., 98.7° F., 98.8° F., 98.9° F., 99.0° F., 99.1° F., 99.2° F., 99.3° F., 99.4° F., 99.5° F., 100° F., 101° F., 102° F., 103° F., 104° F., 105° F., 106° F. or more. The target dosage of a given cannabinoid composition may be determined when a basal body temperature is at least about 97.5° F., 97.6° F., 97.7° F., 97.8° F., 97.9° F., 98.0° F., 98.1° F., 98.2° F., 98.3° F., 98.4° F., 98.5° F., 98.6° F., 98.7° F., 98.8° F., 98.9° F., 99.0° F., 99.1° F., 99.2° F., 99.3° F., 99.4° F., 99.5° F., 100° F., 101° F., 102° F., 103° F., 104° F., 105° F., 106° F., or more. The target dosage of a cannabinoid composition may be determined when a basal body temperature is at most about 106° F., 105° F., 104° F., 103° F., 102° F., 101° F., 100° F., 99.5° F., 99.4° F., 99.3° F., 99.2° F., 99.1° F., 99.0° F., 98.9° F., 98.8° F., 98.7° F., 98.6° F., 98.5° F., 98.4° F., 98.3° F., 98.2° F., 98.1° F., 98.0° F., 97.9° F., 97.8° F., 97.7° F., 97.6° F., 97.5° F., or less. The target dosage of a given cannabinoid composition may be determined for a basal body temperature range from about 97.5° F. to about 98.1° F., about 97.5° F. to about 98.6° F., about 97.5° F. to about 99.5° F., about 97.5° F. to about 100° F., about 97.5° F. to about 102° F., about 97.5° F. to about 104° F., about 97.5° F. to about 106° F., about 98.1° F. to about 98.6° F., about 98.1° F. to about 99.5° F., about 98.1° F. to about 100° F., about 98.1° F. to about 102° F., about 98.1° F. to about 104° F., about 98.1° F. to about 106° F., about 98.6° F. to about 99.5° F., about 98.6° F. to about 100° F., about 98.6° F. to about 102° F., about 98.6° F. to about 104° F., about 98.6° F. to about 106° F., about 99.5° F. to about 100° F., about 99.5° F. to about 102° F., about 99.5° F. to about 104° F., about 99.5° F. to about 106° F., about 100° F. to about 102° F., about 100° F. to about 104° F., about 100° F. to about 106° F., about 102° F. to about 104° F., about 102° F. to about 106° F., or about 104° F. to about 106° F.

The target dosage of a cannabinoid composition may be determined by the measurement of blood sugar concentration. Blood sugar concentration may be defined as the concentration of glucose or another carbohydrate available in the blood stream. A blood sugar concentration may be measured in good health, during a chronic disease state, or during an infective disease state. Blood sugar concentration may be measured as a time series, including more than one measurement, to determine the rate of increase or rate of decrease in blood sugar concentration. A blood sugar concentration may be determined by a blood draw or a personal measurement device. A blood sugar concentration may be measured over a defined period of time, e.g. 30 minutes, 1 hour, 2 hours, or 3 hours. In some instances, a blood sugar concentration may be continuously monitored throughout the administration of a cannabinoid composition. In some instances, a blood sugar concentration may be measured before the administration of a cannabinoid composition. A blood sugar concentration may be subsequently measured after the administration of a cannabinoid composition to determine an effect of the cannabinoid composition on blood sugar concentration. In other instances, a blood sugar concentration may be measured only after the administration of a cannabinoid composition. In some instances, a blood sugar concentration may be repeatedly measured between multiple administrations of a cannabinoid composition until a target dosage or a target blood sugar concentration is achieved. Blood sugar concentration may be measured in a fasting state, after eating, or at a defined interval after eating (e.g. 3 hours after eating). The target dosage of a given cannabinoid composition may be determined when a blood sugar concentration is about 50 milligrams per deciliter (mg/dl), 60 mg/dl, 70 mg/dl, 80 mg/dl, 90 mg/dl, 100 mg/dl, 110 mg/dl, 120 mg/dl, 130 mg/dl, 140 mg/dl, 150 mg/dl, 160 mg/dl, 170 mg/dl, 180 mg/dl, 190 mg/dl, 200 mg/dl, 210 mg/dl, 220 mg/dl, 230 mg/dl, 240 mg/dl, 250 mg/dl, 260 mg/dl, 270 mg/dl, 280 mg/dl, 290 mg/dl, or about 300 mg/dl. The target dosage of a given cannabinoid composition may be determined when a blood sugar concentration is at least about 50 mg/dl, 60 mg/dl, 70 mg/dl, 80 mg/dl, 90 mg/dl, 100 mg/dl, 110 mg/dl, 120 mg/dl, 130 mg/dl, 140 mg/dl, 150 mg/dl, 160 mg/dl, 170 mg/dl, 180 mg/dl, 190 mg/dl, 200 mg/dl, 210 mg/dl, 220 mg/dl, 230 mg/dl, 240 mg/dl, 250 mg/dl, 260 mg/dl, 270 mg/dl, 280 mg/dl, 290 mg/dl, 300 mg/dl, or more. The target dosage of a given cannabinoid composition may be determined when a blood sugar concentration is at most than about 300 mg/dl, 290 mg/dl, 280 mg/dl, 270 mg/dl, 260 mg/dl, 250 mg/dl, 240 mg/dl, 230 mg/dl, 220 mg/dl, 210 mg/dl, 200 mg/dl, 190 mg/dl, 180 mg/dl, 170 mg/dl, 160 mg/dl, 150 mg/dl, 140 mg/dl, 130 mg/dl, 120 mg/dl, 110 mg/dl, 100 mg/dl, 90 mg/dl, 80 mg/dl, 70 mg/dl, 60 mg/dl, or less. The target dosage of a given cannabinoid composition may be determined for a blood sugar concentration from about 50 mg/dl to about 80 mg/dl, about 50 mg/dl to about 120 mg/dl, about 50 mg/dl to about 160 mg/dl, about 50 mg/dl to about 200 mg/dl, about 50 mg/dl to about 240 mg/dl, about 50 mg/dl to about 280 mg/dl, about 50 mg/dl to about 300 mg/dl, about 80 mg/dl to about 120 mg/dl, about 80 mg/dl to about 160 mg/dl, about 80 mg/dl to about 200 mg/dl, about 80 mg/dl to about 240 mg/dl, about 80 mg/dl to about 280 mg/dl, about 80 mg/dl to about 300 mg/dl, about 120 mg/dl to about 160 mg/dl, about 120 mg/dl to about 200 mg/dl, about 120 mg/dl to about 240 mg/dl, about 120 mg/dl to about 280 mg/dl, about 120 mg/dl to about 300 mg/dl, about 160 mg/dl to about 200 mg/dl, about 160 mg/dl to about 240 mg/dl, about 160 mg/dl to about 280 mg/dl, about 160 mg/dl to about 300 mg/dl, about 200 mg/dl to about 240 mg/dl, about 200 mg/dl to about 280 mg/dl, about 200 mg/dl to about 300 mg/dl, about 240 mg/dl to about 280 mg/dl, about 240 mg/dl to about 300 mg/dl, or about 280 mg/dl to about 300 mg/dl.

A target dosage of a given cannabinoid composition may be determined using an invariant personal biomarker. An invariant personal biomarker may be defined as a biometric unique to a given individual that may not vary over a short time period. Examples of invariant personal biomarkers may include height, weight, body fat percentage, lean muscle mass percentage, body mass index, blood type, and resting metabolic rate. A measurement of at least one invariant personal biomarker may occur before the administration of a given cannabinoid composition. An invariant personal biomarker may be used to determine a target dosage of a given cannabinoid composition. An invariant personal biomarker may be used to determine a target dosage of a given cannabinoid composition. For example, a cannabinoid composition may be recommended at a specific amount per pound of body mass. In some instances, a target dosage of a cannabinoid composition may not be determined using an invariant personal biomarker. For example, a cannabinoid composition may be recommended at a dosage that is independent of body mass.

A target dosage of a given cannabinoid composition may be determined by measuring a level of oxidative stress in an individual. A level of oxidative stress may be measured using a variety of techniques in the art. Some of these techniques may include direct measurement of a reduction of reactive oxygen species, measurement of resulting damage to biomolecules, detection of antioxidant levels, or a combination thereof. Reactive oxygen species can include superoxide, peroxyl radical, hydrogen peroxide, hydroxyl radical and peroxynitrite. Probes to detect oxidative stress can be used with a variety of platforms such as fluorescence microscopy, flow cytometry, or microplate analysis. Probes may be dyes that become fluorescent when oxidized or fluoresce to a different color when oxidized. One or more dyes may be used to detect a ratio of reduced to oxidized molecules. To determine a degree of oxidative stress, one or more fluorescent wavelength indicators or dyes may be compared to detect shifts in cellular oxidation. A single wavelength indicator may be used to detect reactive oxygen species.

A level of oxidative stress may be measured by detecting an amount of reactive oxygen species in a cell. Additionally, specific dyes or indicators directed to specific oxidative species may be used to assess cellular oxidative stress. Non-limiting examples of dyes include dihydroethidium, 4-amino-5-methylamino-2′,7′-difluorofluorescein diacetate, and various acetoxymethyl (AM) and acetate ester derivatives of fluorescent indicators. In some instances, an amount of reactive oxygen species measured in a cell may be compared to a threshold amount of reactive oxygen species. For example, the threshold amount of reactive oxygen species can be an amount detected in a subject while the subject is not under oxidative stress.

A level of oxidative stress may be measured by detecting damage to cellular biomolecules. Damaged cellular biomolecules may include damage to protein, deoxyribose nucleic acids (DNA), ribonucleic acids (RNA), lipids or other biomolecules. Biomolecules may be detected using various assays including ELISA, immunoblots, fluorimetry, spectroscopy, and other techniques recognized by one having skill in the art.

A level of oxidative stress may be measured by assaying oxidative damage to nucleic acids. Assaying damage to nucleic acids may include assaying damage to deoxyribose nucleic acids (DNA) or ribonucleic acids (RNA). Examples of assayed biomarkers for oxidative DNA damage include 8-hydroxydeoxyguanosine (8-OHdG), apurinic or apyrimidinic (AP or abasic) sites, cyclobutane pyrimidine dimers (CPD), pyrimidine (6-4) pyrimidone photoproducts (6-4PP), double stranded breaks, or general damage to DNA molecules. Examples of assayed biomarkers for oxidative RNA damage include 8-hydroxyguanosine (8-OHG), apurinic or apyrimidinic (AP or abasic) sites. Biomarkers can be assayed using ELISA, immunofluorescence staining, probes, or other techniques recognized by one having skill in the art.

Measurement of oxidative damage to lipids may be utilized to detect a level of oxidative stress. Oxidative damage to lipids may be measured in a variety of ways. Lipid perodoxidation may be used to measure cellular oxidative stress with respect to the cell membrane. Various techniques involving fluorimetry, ELISA, or immunoblotting may be used to determine lipid perodixation. Dyes or indicators may be configured to specifically detect lipid peroxidation. For live cells one or more indicators may be used. If more than one indicator is used, a ratiometric shift in indicator fluorescence may be indicative of an increase or decrease in oxidative stress. A single indicator may be used to detect lipid-peroxidation derived protein modifications in a fixed cell. Exemplary assays that measure lipid peroxidation may detect 4-HNE (4-hydroxynonenal), 8-iso-Prostaglandin F2a, or malondialdehyde (MDA).

A level of oxidative stress may be ascertained using assays that measure protein carbonyl content, nitrotyrosine, advanced glycation end products, advance oxidation protein end products, benzo(a)pyrene diol epoxide (BPDE) protein adducts, and others recognized by one having skill in the art.

A level of oxidative stress may be detected by assaying the activity of antioxidant molecules. Assaying antioxidant molecules assesses cellular counterbalance to various reactive oxygen species. For instance, the activity of specific antioxidant enzymes such as catalase and superoxide dismutase may be measured. A level of oxidative stress may be ascertained by detecting reduced glutathione (GSH) levels, which can be an indication of redox potential and a cell's ability to prevent oxidative stress. Some non-limiting examples of dyes that may be used to detect GSH levels include monoclorobimane (mBCl) and monobromobimane (mBBr). Ratiometric fluorescence assays may be used to assess the balance between reactive oxygen species and antioxidants.

A level of oxidative stress may be measured in cells sampled from a subject. A level of oxidative stress may be measured within a variety of biological sample types. For instance, a level of oxidative stress may be measured in cells from a biological sample type, including saliva, blood, sweat, serum, cerebrospinal fluid, plasma, urine, or any suspension of cells from a subject.

A cannabinoid composition may contain a calibrated amount of microencapsulated cannabinoids. Alternatively, a cannabinoid composition may contain a calibrated amount of non-encapsulated cannabinoids. The concentration of cannabinoids may vary depending upon the precise formulation and application of the cannabinoid composition. A cannabinoid composition may contain at least about 1 nanogram per milliliter (ng/ml), 10 ng/ml, 100 ng/ml, 500 ng/ml, 1 microgram per milliliter (μg/ml), 5 μg/ml, 10 μg/ml, 15 μg/ml, 20 μg/ml, 25 μg/ml, 30 μg/ml, 35 μg/ml, 40 μg/ml, 45 μg/ml, 50 μg/ml, 55 μg/ml, 60 μg/ml, 65 μg/ml, 70 μg/ml, 75 μg/ml, 80 μg/ml, 85 μg/ml, 90 μg/ml, 95 μg/ml, 100 μg/ml, 105 μg/ml, 110 μg/ml, 115 μg/ml, 120 μg/ml, 125 μg/ml, 130 μg/ml, 135 μg/ml, 140 μg/ml, 145 μg/ml, 150 μg/ml, 155 μg/ml, 160 μg/ml, 165 μg/ml, 170 μg/ml, 175 μg/ml, 180 μg/ml, 185 μg/ml, 190 μg/ml, 195 μg/ml, or at least about 200 μg/ml of the microencapsulated cannabinoids per weight of the cannabinoid composition. A cannabinoid composition may contain at most about 200 μg/ml, 195 μg/ml, 190 μg/ml, 185 μg/ml, 180 μg/ml, 175 μg/ml, 170 μg/ml, 165 μg/ml, 160 μg/ml, 155 μg/ml, 150 μg/ml, 145 μg/ml, 140 μg/ml, 135 μg/ml, 130 μg/ml, 125 μg/ml, 120 μg/ml, 115 μg/ml, 110 μg/ml, 105 μg/ml, 100 μg/ml, 95 μg/ml, 90 μg/ml, 85 μg/ml, 80 μg/ml, 75 μg/ml, 70 μg/ml, 65 μg/ml, 60 μg/ml, 55 μg/ml, 50 μg/ml, 45 μg/ml, 40 μg/ml, 35 μg/ml, 30 μg/ml, 25 μg/ml, 20 μg/ml, 15 μg/ml, 10 μg/ml, 5 μg/ml, 1 μg/ml, 500 ng/ml, 100 ng/ml, 10 ng/ml, 1 ng/ml, or less of the microencapsulated cannabinoids per weight of the cannabinoid composition.

An amount of the microcapsules (e.g., a plurality of microcapsules comprising at least one cannabinoid compound) in the cannabinoid composition may be at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%1, 3%, 14%, 15%, 16%, 17%, 18%, 19%, 20%, 25%, 30%, 35%, 40%, 4%5% 50%, or more by weight of the cannabinoid composition. The amount of the microcapsules in the cannabinoid composition may be at most about 50%, 45%, 40%, 35%, 30%, 25%, 20%, 19%, 18%, 17%, 16%, 15%, 4, 13%, %12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.10%, or less by weight of the cannabinoid composition. The amount of the microcapsules in the cannabinoid composition may be at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or more by volume of the cannabinoid composition. The amount of the microcapsules in the cannabinoid composition may be at most about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or less by volume of the cannabinoid composition

A cannabinoid composition may be delivered at a particular concentration and a particular volume to achieve a desired dosage of cannabinoids. The dosage volume of a cannabinoid composition may vary depending upon the concentration and application of the cannabinoid composition. A cannabinoid composition dosage may have a volume of at least about 1 microliter (μl), 10 μl, 100 μl, 200 μl, 300 μl, 400 μl, 500 μl, 600 μl, 700 μl, 800 μl, 900 μl, 1 milliliter (ml), 2 ml, 3 ml, 4 ml, 5 ml, 6 ml, 7 ml, 8 ml, 9 ml, 10 ml, 11 ml, 12 ml, 13 ml, 14 ml, 15 ml, 16 ml, 17 ml, 18 ml, 19 ml, 20 ml, 21 ml, 22 ml, 23 ml, 24 ml, 25 ml, 26 ml, 27 ml, 28 ml, 29 ml, 30 ml, 31 ml, 32 ml, 33 ml, 34 ml, 35 ml, 36 ml, 37 ml, 38 ml, 39 ml, 40 ml, 41 ml, 42 ml, 43 ml, 44 ml, 45 ml, 46 ml, 47 ml, 48 ml, 49 ml, 50 ml, 51 ml, 52 ml, 53 ml, 54 ml, 55 ml, 56 ml, 57 ml, 58 ml, 59 ml, 60 ml, 61 ml, 62 ml, 63 ml, 64 ml, 65 ml, 66 ml, 67 ml, 68 ml, 69 ml, 70 ml, 71 ml, 72 ml, 73 ml, 74 ml, 75 ml, 76 ml, 77 ml, 78 ml, 79 ml, 80 ml, 81 ml, 82 ml, 83 ml, 84 ml, 85 ml, 86 ml, 87 ml, 88 ml, 89 ml, 90 ml, 91 ml, 92 ml, 93 ml, 94 ml, 95 ml, 96 ml, 97 ml, 98 ml, 99 ml, 100 ml, 110 ml, 120 ml, 130 ml, 140 ml, 150 ml, 160 ml, 170 ml, 180 ml, 190 ml, 200 ml, or more. A cannabinoid composition dosage may have a volume of at most about 200 ml, 190 ml, 180 ml, 170 ml, 160 ml, 150 ml, 140 ml, 130 ml, 120 ml, 110 ml, 100 ml, 99 ml, 98 ml, 97 ml, 96 ml, 95 ml, 94 ml, 93 ml, 92 ml, 91 ml, 90 ml, 89 ml, 88 ml, 87 ml, 86 ml, 85 ml, 84 ml, 83 ml, 82 ml, 81 ml, 80 ml, 79 ml, 78 ml, 77 ml, 76 ml, 75 ml, 74 ml, 73 ml, 72 ml, 71 ml, 70 ml, 69 ml, 68 ml, 67 ml, 66 ml, 65 ml, 64 ml, 63 ml, 62 ml, 61 ml, 60 ml, 59 ml, 58 ml, 57 ml, 56 ml, 55 ml, 54 ml, 53 ml, 52 ml, 51 ml, 50 ml, 49 ml, 48 ml, 47 ml, 46 ml, 45 ml, 44 ml, 43 ml, 42 ml, 41 ml, 40 ml, 39 ml, 38 ml, 37 ml, 36 ml, 35 ml, 34 ml, 33 ml, 32 ml, 31 ml, 30 ml, 29 ml, 28 ml, 27 ml, 26 ml, 25 ml, 24 ml, 23 ml, 22 ml, 21 ml, 20 ml, 19 ml, 18 ml, 17 ml, 16 ml, 15 ml, 14 ml, 13 ml, 12 ml, 11 ml, 10 ml, 9 ml, 8 ml, 7 ml, 6 ml, 5 ml, 4 ml, 3 ml, 2 ml, 1 ml, 900 μl, 800 μl, 700 μl, 600 μl, 500 μl, 400 μl, 300 μl, 200 μl, 100 μl, 10 μl, 1 μl, or less. A cannabinoid composition dosage may occur in a range from about 1 μl to about 100 μl, about 1 μl to about 500 μl, about 1 μl to about 1 ml, about 1 μl to about 5 ml, about 1 μl to about 10 ml, about 1 μl to about 15 ml, about 1 μl to about 20 ml, about 1 μl to about 25 ml, about 1 μl to about 50 ml, about 1 μl to about 100 ml, about 1 μl to about 150 ml, about 1 μl to about 200 ml, about 100 μl to about 500 μl, about 100 μl to about 1 ml, about 100 μl to about 5 ml, about 100 μl to about 10 ml, about 100 μl to about 15 ml, about 100 μl to about 20 ml, about 100 μl to about 25 ml, about 100 μl to about 50 ml, about 100 μl to about 100 ml, about 100 μl to about 150 ml, about 100 μl to about 200 ml, about 500 μl to about 1 ml, about 500 μl to about 5 ml, about 500 μl to about 10 ml, about 500 μl to about 15 ml, about 500 μl to about 20 ml, about 500 μl to about 25 ml, about 500 μl to about 50 ml, about 500 μl to about 100 ml, about 500 μl to about 150 ml, about 500 μl to about 200 ml, about 1 ml to about 5 ml, about 1 ml to about 10 ml, about 1 ml to about 15 ml, about 1 ml to about 20 ml, about 1 ml to about 25 ml, about 1 ml to about 50 ml, about 1 ml to about 100 ml, about 1 ml to about 150 ml, about 1 ml to about 200 ml, about 5 ml to about 10 ml, about 5 ml to about 15 ml, about 5 ml to about 20 ml, about 5 ml to about 25 ml, about 5 ml to about 50 ml, about 5 ml to about 100 ml, about 5 ml to about 150 ml, about 5 ml to about 200 ml, about 10 ml to about 15 ml, about 10 ml to about 20 ml, about 10 ml to about 25 ml, about 10 ml to about 50 ml, about 10 ml to about 100 ml, about 10 ml to about 150 ml, about 10 ml to about 200 ml, about 15 ml to about 20 ml, about 15 ml to about 25 ml, about 15 ml to about 50 ml, about 15 ml to about 100 ml, about 15 ml to about 150 ml, about 15 ml to about 200 ml, about 20 ml to about 25 ml, about 20 ml to about 50 ml, about 20 ml to about 100 ml, about 20 ml to about 150 ml, about 20 ml to about 200 ml, about 25 ml to about 50 ml, about 25 ml to about 100 ml, about 25 ml to about 150 ml, about 25 ml to about 200 ml, about 50 ml to about 100 ml, about 50 ml to about 150 ml, about 50 ml to about 200 ml, about 100 ml to about 150 ml, about 100 ml to about 200 ml, or about 150 ml to about 200 ml.

A cannabinoid composition may have associated chemical and physical properties based upon its constituent components. The chemical and physical properties of a cannabinoid composition may include density, viscosity, pH, taste, and flavor. In the present disclosure, “taste” generally refers to the specific sensory perception tied to the chemical composition of an ingested composition. Tastes may include salty (ionic content, especially of sodium), sweet (carbohydrate content), bitter (the presence of amines and other compounds that bind bitter taste receptors), sour (acidic compounds and other compounds that bind sour receptors), and savory (protein-related compounds) characterizations. In the present disclosure, “flavor” generally refers to the complex combinations of tastes that are distinct to a particular ingested composition, e.g., the flavor of coffee versus the flavor of chocolate is distinguished by the phytochemical differences between coffee and cacao plants.

In some aspects, the chemical and physical properties of a cannabinoid composition may be used to determine an individual's target dosage. In some examples, a cannabinoid composition may be formulated with a particular pH. A cannabinoid composition may have a pH of about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or about 9.0. A cannabinoid composition may have a pH of no greater than about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, or about 4.0. A cannabinoid composition may have a pH of no less than about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or about 9.0. A cannabinoid composition may have a pH range from about 4.0 to about 4.5, about 4.0 to about 5.0, about 4.0 to about 5.5, about 4.0 to about 6.0, about 4.0 to about 6.5, about 4.0 to about 7.0, about 4.0 to about 7.5, about 4.0 to about 8.0, about 4.0 to about 8.5, about 4.0 to about 9.0, about 4.5 to about 5.0, about 4.5 to about 5.5, about 4.5 to about 6.0, about 4.5 to about 6.5, about 4.5 to about 7.0, about 4.5 to about 7.5, about 4.5 to about 8.0, about 4.5 to about 8.5, about 4.5 to about 9.0, about 5.0 to about 5.5, about 5.0 to about 6.0, about 5.0 to about 6.5, about 5.0 to about 7.0, about 5.0 to about 7.5, about 5.0 to about 8.0, about 5.0 to about 8.5, about 5.0 to about 9.0, about 5.5 to about 6.0, about 5.5 to about 6.5, about 5.5 to about 7.0, about 5.5 to about 7.5, about 5.5 to about 8.0, about 5.5 to about 8.5, about 5.5 to about 9.0, about 6.0 to about 6.5, about 6.0 to about 7.0, about 6.0 to about 7.5, about 6.0 to about 8.0, about 6.0 to about 8.5, about 6.0 to about 9.0, about 6.5 to about 7.0, about 6.5 to about 7.5, about 6.5 to about 8.0, about 6.5 to about 8.5, about 6.5 to about 9.0, about 7.0 to about 7.5, about 7.0 to about 8.0, about 7.0 to about 8.5, about 7.0 to about 9.0, about 7.5 to about 8.0, about 7.5 to about 8.5, about 7.5 to about 9.0, about 8.0 to about 8.5, about 8.0 to about 9.0, or about 8.5 to about 9.0.

The taste and flavor of a cannabinoid composition may vary depending upon its chemical composition. The presence of particular compounds may give a cannabinoid composition a salty, sour, bitter, or sweet taste. The flavor of a cannabinoid composition may be altered or enhanced by the addition of ingredients such as essential oils, herbal ingredients, and food oils. In some examples, the target dosage of a cannabinoid may be determined by the perception of the taste and flavor of a cannabinoid composition during oral ingestion. In a particular example, the perception of the taste of a cannabinoid composition may change when the target dosage has been achieved.

Taste may be linked to the biochemical contact between sensory receptors (taste buds) and particular compounds within ingested solids and liquids. Each taste bud may be capable of distinguishing at least five distinct tastes, including saltiness, sourness, bitterness, sweetness, and savoryness. Without being bound to a particular physical theory, it can be generally stated that the perception of particular tastes are linked to the activation of different physical mechanisms within a taste bud when the taste bud is physically contacted by a molecule solvated within saliva. The physical mechanism, e.g., the internal release of calcium ions, triggers a signal within a nerve linked to the taste bud, transmitting a taste signal to the brain. Simultaneous or temporally-varying activation of multiple taste mechanisms give rise to the complex signals that are resolved in the brain as flavors.

Taste buds are typically located in the mouth, especially on the surfaces of the tongue. A human mouth may contain between about 2000 and about 8000 taste buds. Taste buds can be found on the ventral, dorsal, and lateral sides of the tongue. The presence of taste buds on the dorsal and lateral sides of the tongue means that tastes can be detected when solids or liquids are only brought in contact with the sublingual region of the mouth. Taste perception may be influenced by other factors, including age, weight, hydration state, disease state, hormonal state (e.g. hunger is hormonally-linked), oral microbiome, and prior ingestion of solids and liquids. In some aspects, saliva may play an important role in solvating particular chemical compounds such that they are perceived by the taste buds.

Saliva may contain a complex mixture of chemical compounds, macromolecules, and cells. Saliva may comprise water, hydrogen peroxide, and ionic compounds such as sodium, potassium, calcium, magnesium, chloride, bicarbonate, phosphate, thiocyanate, and iodine. Saliva may comprise macromolecules such as mucopolysaccharides, glycoproteins, epidermal growth factor, and opiorphin. Saliva may comprise digestive enzymes such as α-amylase, lingual lipase, kallikrein, salivary acid phosphatase A, salivary acid phosphatase B, N-acetylmuramoyl-L-alanine amidase, NADPH dehydrogenase, superoxide dismutase, glutathione transferase, aldehyde dehydrogenase 3, haptocorrin, and glucose-6-phospate isomerase. Saliva may comprise antimicrobial proteins such as lysozyme, salivary lactoperoxidase, lactoferrin, and immunoglobulin A. Saliva may comprise human cells such as leukocytes and epithelial cells. Saliva may comprise non-human cells such as bacterial and fungal cells. The oral bacterial microbiome may include species from the genera Actinomyces, Bacteroides, Bifidobacterium, Eubacterium, Fusobacterium, Lactobacillus, Leptotrichia, Peptococcus, Peptostreptococcus, Propionobacterium, Selenomonas, Streptococcus, Treponema, and Veillonella. The oral fungal microbiome may include species from the genera Candida, Cladosporium, Aspergillus, Fusarium, Glomus, Alternaria, Penicillium, and Cryptococcus.

Saliva may be characterized by a particular pH. Saliva may have a pH of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or about 9.0. Saliva may have a pH of no greater than about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, or about 5.0. Saliva may have a pH of no less than about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or about 9.0. Saliva may have a pH range from about 5.0 to about 5.5, about 5.0 to about 6.0, about 5.0 to about 6.5, about 5.0 to about 7.0, about 5.0 to about 7.5, about 5.0 to about 8.0, about 5.0 to about 8.5, about 5.0 to about 9.0, about 5.5 to about 6.0, about 5.5 to about 6.5, about 5.5 to about 7.0, about 5.5 to about 7.5, about 5.5 to about 8.0, about 5.5 to about 8.5, about 5.5 to about 9.0, about 6.0 to about 6.5, about 6.0 to about 7.0, about 6.0 to about 7.5, about 6.0 to about 8.0, about 6.0 to about 8.5, about 6.0 to about 9.0, about 6.5 to about 7.0, about 6.5 to about 7.5, about 6.5 to about 8.0, about 6.5 to about 8.5, about 6.5 to about 9.0, about 7.0 to about 7.5, about 7.0 to about 8.0, about 7.0 to about 8.5, about 7.0 to about 9.0, about 7.5 to about 8.0, about 7.5 to about 8.5, about 7.5 to about 9.0, about 8.0 to about 8.5, about 8.0 to about 9.0, or about 8.5 to about 9.0.

The overall pH of the mouth may be influenced by several factors including the pH of saliva, the chemical composition of recently ingested solids or liquids, and secretions by oral microbiota. In some aspects, the pH of the mouth may become more acidic via ingestion of acidic solids or liquids, e.g. citrus fruits or vinegars. In other aspects, the pH of the mouth may become more basic via ingestion of basic solids or liquids, e.g. yogurt or milk. In some aspects, a solid or liquid may be consumed to alter the pH of the mouth. In some aspects, the mouth may be rinsed with a solution to optimize the pH of the mouth. The mouth may be characterized by a particular pH. The mouth may have a pH of about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or about 9.0. The mouth may have a pH of no greater than about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, or about 4.0. The mouth may have a pH of no less than about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or about 9.0. The mouth may have a pH range from about 4.0 to about 4.5, about 4.0 to about 5.0, about 4.0 to about 5.5, about 4.0 to about 6.0, about 4.0 to about 6.5, about 4.0 to about 7.0, about 4.0 to about 7.5, about 4.0 to about 8.0, about 4.0 to about 8.5, about 4.0 to about 9.0, about 4.5 to about 5.0, about 4.5 to about 5.5, about 4.5 to about 6.0, about 4.5 to about 6.5, about 4.5 to about 7.0, about 4.5 to about 7.5, about 4.5 to about 8.0, about 4.5 to about 8.5, about 4.5 to about 9.0, about 5.0 to about 5.5, about 5.0 to about 6.0, about 5.0 to about 6.5, about 5.0 to about 7.0, about 5.0 to about 7.5, about 5.0 to about 8.0, about 5.0 to about 8.5, about 5.0 to about 9.0, about 5.5 to about 6.0, about 5.5 to about 6.5, about 5.5 to about 7.0, about 5.5 to about 7.5, about 5.5 to about 8.0, about 5.5 to about 8.5, about 5.5 to about 9.0, about 6.0 to about 6.5, about 6.0 to about 7.0, about 6.0 to about 7.5, about 6.0 to about 8.0, about 6.0 to about 8.5, about 6.0 to about 9.0, about 6.5 to about 7.0, about 6.5 to about 7.5, about 6.5 to about 8.0, about 6.5 to about 8.5, about 6.5 to about 9.0, about 7.0 to about 7.5, about 7.0 to about 8.0, about 7.0 to about 8.5, about 7.0 to about 9.0, about 7.5 to about 8.0, about 7.5 to about 8.5, about 7.5 to about 9.0, about 8.0 to about 8.5, about 8.0 to about 9.0, or about 8.5 to about 9.0.

Endocannabinoids, produced naturally in individuals, bind with cannabinoid receptors (e.g., CB1 receptors) to help regulate various functions and aspects of health (e.g., homeostasis, appetite, etc.) of the body. Endocannabinoid stimuli may specifically and selectively trigger or enhance a response to the sweet taste, with little or no change to other tastes including salty, bitter, sour, and savory. Exogenous cannabinoid compounds, such as one or more cannabinoid compounds included in the cannabinoid composition of the present disclosure, may bind with such cannabinoid receptors to facilitate regulation of the various functions and aspects of health of the individual by supplementing the endocannabinoid levels. A perception or change of taste with respect to the cannabinoid composition may be indicative of the activity of the cannabinoid receptors of the individual, and therapeutic efficacy of the cannabinoid composition to regulate veracious functions and aspects of the body.

Accordingly, a target dosage of a cannabinoid composition may be determined by changes in the perception of the taste of the composition. In some examples, the cannabinoid composition may be administered in a sufficient quantity for the individual to perceive a change from a first taste to a second taste. The first taste may be chosen from the group consisting of salty, bitter, sour, sweet, and savory. The second taste will be any taste other than the first taste, and may be chosen from the group consisting of salty, bitter, sour, sweet, and savory. In a particular example, an individual may perceive a first bitter taste upon initial oral application of the cannabinoid composition and may experience a second sweet taste when a target dosage has been achieved. In some examples, the cannabinoid composition may be administered in a sufficient quantity for the individual to perceive a change from a first flavor to a second flavor. In a particular example, an individual may perceive a first bitter flavor, such as an herbal flavor, upon initial oral application of the cannabinoid composition and may experience a second sweet flavor, such as a honey flavor, when a target dosage has been achieved, i.e., when a sufficient amount of cannabinoid compounds comprising the exogeneous cannabinoid compounds from the applied cannabinoid composition of the present disclosure binds and/or activates the cannabinoid receptors of the individual.

In some aspects, a target dosage for a cannabinoid composition may be determined by oral application of the composition. In some aspects, solid or semi-solid cannabinoid compositions may be applied to the roof of the mouth or the tongue. In some aspects, liquid cannabinoid compositions may be applied to the tongue or sublingually. In some aspects, the cannabinoid composition may be applied in measured amounts or volumes. Additional volumes, that may or may not be the same volumes, may be applied to the specified area of the mouth until a change in taste or flavor is perceived. The cumulative volume at which a complete change in the taste or flavor of the cannabinoid composition is detected, or perceived by an individual, may correspond to the target dosage of a cannabinoid composition for the individual. A liquid cannabinoid composition may be applied by a dropper or other device that allows accurate and reliable measurement of liquid volumes. A liquid cannabinoid composition may be dispensed in a fixed test volume. A fixed test volume may comprise a full dropper. A fixed test volume may comprise a single drop from a dropper.

For a fixed test volume (e.g., 10 milliliters (ml)), one or more volumes of the cannabinoid composition may be necessary to achieve the target dosage. A fixed test volume may have a volume of about 0.1 ml, 0.5 ml, 1 ml, 2 ml, 3 ml, 4 ml, 5 ml, 6 ml, 7 ml, 8 ml, 9 ml, 10 ml, 15 ml, 20 ml, 25 ml, 50 ml, or more. A fixed test volume may have a volume of at least about 0.1 ml, 0.5 ml, 1 ml, 2 ml, 3 ml, 4 ml, 5 ml, 6 ml, 7 ml, 8 ml, 9 ml, 10 ml, 15 ml, 20 ml, 25 ml, or at least about 50 ml or more. A fixed test volume may have a volume of no more than about 50 ml, 25 ml, 20 ml, 15 ml, 10 ml, 9 ml, 8 ml, 7 ml, 6 ml, 5 ml, 4 ml, 3 ml, 2 ml. 1 ml, 0.5 ml, or no more than about 0.1 ml or less.

A target dosage may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 fixed test volumes. A target dosage may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or at least about 50 fixed test volumes. A target dosage may comprise no more than about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or no more than about 1 fixed test volume. A target dosage may comprise from about 1 to about 5 fixed test volumes, about 1 to about 10 fixed test volumes, about 1 to about 15 fixed test volumes, about 1 to about 20 fixed test volumes, about 1 to about 25 fixed test volumes, about 1 to about 30 fixed test volumes, about 1 to about 35 fixed test volumes, about 1 to about 40 fixed test volumes, about 1 to about 45 fixed test volumes, about 1 to about 50 fixed test volumes, about 5 to about 10 fixed test volumes, about 5 to about 15 fixed test volumes, about 5 to about 20 fixed test volumes, about 5 to about 25 fixed test volumes, about 5 to about 30 fixed test volumes, about 5 to about 35 fixed test volumes, about 5 to about 40 fixed test volumes, about 5 to about 45 fixed test volumes, about 5 to about 50 fixed test volumes, about 10 to about 15 fixed test volumes, about 10 to about 20 fixed test volumes, about 10 to about 25 fixed test volumes, about 10 to about 30 fixed test volumes, about 10 to about 35 fixed test volumes, about 10 to about 40 fixed test volumes, about 10 to about 45 fixed test volumes, about 10 to about 50 fixed test volumes, about 15 to about 20 fixed test volumes, about 15 to about 25 fixed test volumes, about 15 to about 30 fixed test volumes, about 15 to about 35 fixed test volumes, about 15 to about 40 fixed test volumes, about 15 to about 45 fixed test volumes, about 15 to about 50 fixed test volumes, about 20 to about 25 fixed test volumes, about 20 to about 30 fixed test volumes, about 20 to about 35 fixed test volumes, about 20 to about 40 fixed test volumes, about 20 to about 45 fixed test volumes, about 20 to about 50 fixed test volumes, about 25 to about 30 fixed test volumes, about 25 to about 35 fixed test volumes, about 25 to about 40 fixed test volumes, about 25 to about 45 fixed test volumes, about 25 to about 50 fixed test volumes, about 30 to about 35 fixed test volumes, about 30 to about 40 fixed test volumes, about 30 to about 45 fixed test volumes, about 30 to about 50 fixed test volumes, about 35 to about 40 fixed test volumes, about 35 to about 45 fixed test volumes, about 35 to about 50 fixed test volumes, about 40 to about 45 fixed test volumes, about 40 to about 50 fixed test volumes, or about 45 to about 50 fixed test volumes.

In some aspects, a cannabinoid composition may be applied orally in fixed test volumes until a partial change in taste or flavor is perceived, and then smaller test volumes may be applied orally until a full change in taste or flavor is perceived by the individual. The smaller test volumes may be fixed. A smaller test volume may be about 5%, 10%, 15%, 20%, 25%, 30%, 35%, 40%, 45%, 50%, 55%, 60%, 65%, 70%, 75%, 80%, 85%, 90%, 95%, 99% or more of a full test volume. A smaller test volume may be at least about 5%, 10%, 15%, 20%, 25%, 30%, 35%, 40%, 45%, 50%, 55%, 60%, 65%, 70%, 75%, 80%, 85%, 90%, 95%, or at least about 99% or more of a full test volume. A smaller test volume may be no more than about 99%, 95%, 90%, 85%, 80%, 75%, 70%, 65%, 60%, 55%50%, 45%, 40%, 35%, 30%, 25%, 20%1, 5%, 10%, or no more than about 5% of a full test volume. In some instances, varying smaller volumes (e.g., increasingly smaller volumes) may be applied as the partial change in taste or flavor approaches a full change in taste or flavor. Such smaller volumes may be recorded with each application to determine a cumulative volume at the point of detection, or perception, of the full change in taste or flavor.

In some aspects, a target dosage may be determined by the addition of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 smaller fixed test volumes. A target dosage may be determined by the addition of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or at least about 50 smaller fixed test volumes. A target dosage may be determined by the addition of no more than about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or no more than about 1 smaller fixed test volume.

A target dosage may be determined by applying (e.g., orally administering) a plurality of test volumes of the cannabinoid composition to the individual. The plurality of test volumes may be the same or different. The plurality of test volumes may be applied to the individual within minutes of each other, hours, days, or even weeks. The intervals between the plurality of test volumes may be regular (i.e., foxed) or irregular. In some cases, the plurality of test volumes may be applied to the individual at an interval of at least about one test volume per every 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 20 minutes, 25 minutes, 30 minutes, 40 minutes, 50 minutes, 60 minutes, 2 hours, 3 hours, 4 hours, 5 hours, 6 hours, 7 hours, 8 hours, 9 hours, 10 hours, 11 hours, 12 hours, 16 hours, 20 hours, 24 hours, or more. The plurality of test volumes may be applied to the individual at an interval of at most about one test volume per every 24 hours, 20 hours, 16 hours, 12 hours, 11 hours, 10 hours, 9 hours, 8 hours, 7 hours, 6 hours, 5 hours, 4 hours, 3 hours, 2 hours, 60 minutes, 50 minutes, 40 minutes, 30 minutes, 25 minutes, 20 minutes, 15 minutes, 14 minutes, 13 minutes, 12 minutes, 11 minutes, 10 minutes, 9 minutes, 8 minutes, 7 minutes, 6 minutes, 5 minutes, 4 minutes, 3 minutes, 2 minutes, 1 minute, or less.

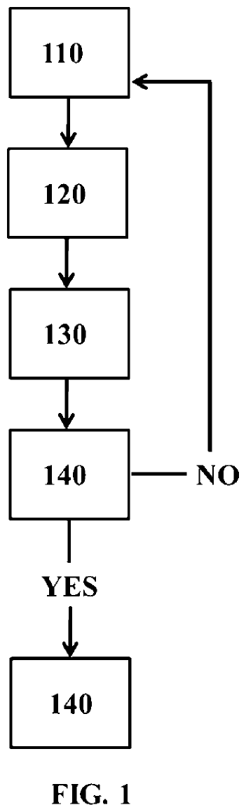

FIG. 1 depicts a method for approximately determining the target dosage of a cannabinoid composition. In the first operation 110, a first fixed test volume of a cannabinoid composition is applied to a region of the mouth. In the second operation 120, the individual perceives the taste of the cannabinoid composition to form an initial taste determination. For example, the initial taste determination may be characterized as one or more members of the taste group consisting of salty, bitter, sweet, sour, and savory. In the third operation 130, a second fixed test volume of the cannabinoid composition is applied to the region of the mouth. In the fourth operation 140, the individual perceives the taste of the cannabinoid composition to form a subsequent taste determination. The subsequent taste determination may be characterized as one or more members of the taste group consisting of salty, bitter, sweet, sour, and savory. The individual evaluates whether there has been a change between the initial taste determination and the subsequent taste determination. In some instances, the change may be identified if there is at least one change in the one or more members of the taste group between the initial taste determination and the subsequent taste determination. If the change is identified, sufficient dosage has been achieved, and a cumulative volume (e.g., sum of the first test volume and the second test volume) may be indicative of the target dosage for the individual. If the change is not identified, the first through fourth operations are repeated until a change is detected. The total volume of the orally-applied cannabinoid composition is indicative of the approximate target dosage 150 for the cannabinoid composition.

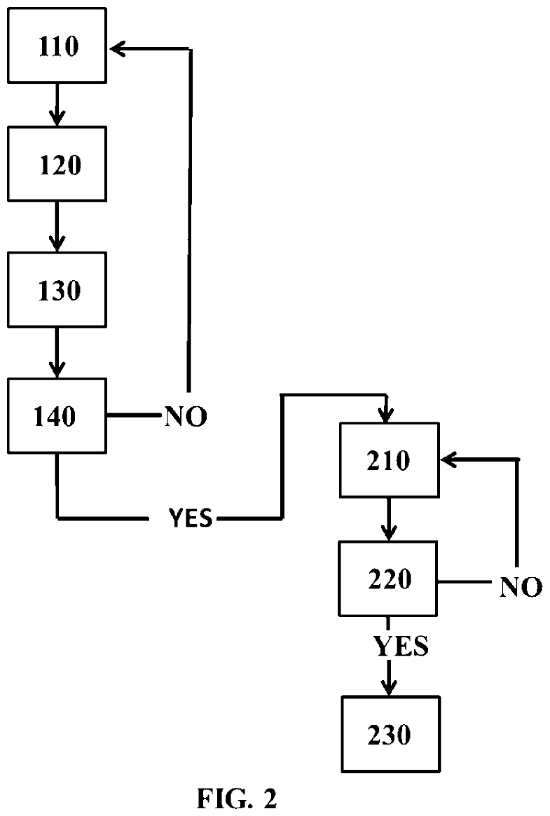

FIG. 2 depicts a refined method for determining a more precise target dosage of a cannabinoid composition. The method of FIG. 1 is performed until an approximate target dosage 150 is achieved. In a fifth operation 210, the individual applies a smaller fixed test volume of the cannabinoid composition. In a sixth operation 220, the individual perceives the taste and determines if it exactly matches the target taste of the cannabinoid composition. If the taste does not match, smaller test volumes of cannabinoid are added until the target taste is achieved. The total volume of the orally-applied cannabinoid composition is the exact target dosage 230 for the cannabinoid composition.

The determination of a target cannabinoid dosage may be preceded or followed by additional operations. A rinse may be used to cleanse the mouth, adjust the oral pH, remove food debris, provide an initial taste or flavor, or otherwise prepare the mouth for dosage determination. A rinse or drink may be used after the dosage determination to remove the flavor of the cannabinoid composition. The rinse or drink may be used once or repeatedly until a particular oral state is achieved, e.g. a pH of 7.0. A rinsing or cleansing process may be repeated at least 1, 2, 3, 4, 5, 6, 7, 8, 9, or at least 10 times. A rinsing or cleansing process may be repeated no more than about 10, 9, 8, 7, 6, 5, 4, 3, 2, or no more than 1 time. The rinse or drink may be formulated to have a particular taste, flavor, composition or pH. The rinse or drink may have a pH of about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or about 9.0. The rinse or drink may have a pH of no greater than about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, or about 4.0. The rinse or drink may have a pH of no less than about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or about 9.0. The rinse or drink may have a pH range from about 4.0 to about 4.5, about 4.0 to about 5.0, about 4.0 to about 5.5, about 4.0 to about 6.0, about 4.0 to about 6.5, about 4.0 to about 7.0, about 4.0 to about 7.5, about 4.0 to about 8.0, about 4.0 to about 8.5, about 4.0 to about 9.0, about 4.5 to about 5.0, about 4.5 to about 5.5, about 4.5 to about 6.0, about 4.5 to about 6.5, about 4.5 to about 7.0, about 4.5 to about 7.5, about 4.5 to about 8.0, about 4.5 to about 8.5, about 4.5 to about 9.0, about 5.0 to about 5.5, about 5.0 to about 6.0, about 5.0 to about 6.5, about 5.0 to about 7.0, about 5.0 to about 7.5, about 5.0 to about 8.0, about 5.0 to about 8.5, about 5.0 to about 9.0, about 5.5 to about 6.0, about 5.5 to about 6.5, about 5.5 to about 7.0, about 5.5 to about 7.5, about 5.5 to about 8.0, about 5.5 to about 8.5, about 5.5 to about 9.0, about 6.0 to about 6.5, about 6.0 to about 7.0, about 6.0 to about 7.5, about 6.0 to about 8.0, about 6.0 to about 8.5, about 6.0 to about 9.0, about 6.5 to about 7.0, about 6.5 to about 7.5, about 6.5 to about 8.0, about 6.5 to about 8.5, about 6.5 to about 9.0, about 7.0 to about 7.5, about 7.0 to about 8.0, about 7.0 to about 8.5, about 7.0 to about 9.0, about 7.5 to about 8.0, about 7.5 to about 8.5, about 7.5 to about 9.0, about 8.0 to about 8.5, about 8.0 to about 9.0, or about 8.5 to about 9.0.

A target dosage of a cannabinoid composition may be administered once or more per day. A target dosage of a cannabinoid composition may be administered about 1, 2, 3, 4, 5, 6, 7, 8, 9, or about 10 times per day. A target dosage of a cannabinoid composition may be administered at least about 1, 2, 3, 4, 5, 6, 7, 8, 9, or about 10 times per day. A target dosage of a cannabinoid composition may be administered no more than about 10, 9, 8, 7, 6, 5, 4, 3, 2, or no more than about 1 time per day. A target dosage for a given cannabinoid composition may be measured daily, weekly, or monthly. A target dosage for a given cannabinoid composition may change depending upon changes in an individual health and environment. The target dosage for a given individual may be linked to certain biomarkers that can be monitored as evidence of changes in an individual's health or environmental state.

A cannabinoid composition may be packaged as a kit comprising the cannabinoid composition, an applicator device capable of dispensing the cannabinoid composition in one or more fixed volumes, and instructions for determining the target dosage of the cannabinoid composition. The provided instructions may provide all relevant information including the expected initial taste or flavor of the cannabinoid composition, and the target taste or flavor when the target dosage has been achieved. The kit may comprise an applicator device such as a medicine dropper or measuring cup. An applicator device such as a medicine dropper may be capable of dispensing in more than one fixed test volume. For example, a medicine dropper may have a large fixed test volume from dispensing the entire dropper volume, or a smaller fixed test volume from dispensing a single drop. A medicine dropper may have a fixed total volume of about 1, 2, 3, 4, 5, 6, 7, 8, 9, or about 10 ml of liquid. A medicine dropper may have a fixed total volume of at least about 1, 2, 3, 4, 5, 6, 7, 8, 9, or about 10 ml of liquid. A medicine dropper may have a fixed total volume of no more than about 10, 9, 8, 7, 6, 5, 4, 3, 2, or no more than about 1 ml. A medicine dropper may have a fixed total volume from about 1 ml to 2 ml, 1 ml to about 3 ml, 1 ml to about 4 ml, 1 ml to about 5 ml, 1 ml to about 6 ml, 1 ml to about 7 ml, 1 ml to about 8 ml, 1 ml to about 9 ml, 1 ml to about 10 ml, 2 ml to about 3 ml, 2 ml to about 4 ml, 2 ml to about 5 ml, 2 ml to about 6 ml, 2 ml to about 7 ml, 2 ml to about 8 ml, 2 ml to about 9 ml, 2 ml to about 10 ml, 3 ml to about 4 ml, 3 ml to about 5 ml, 3 ml to about 6 ml, 3 ml to about 7 ml, 3 ml to about 8 ml, 3 ml to about 9 ml, 3 ml to about 10 ml, 4 ml to about 5 ml, 4 ml to about 6 ml, 4 ml to about 7 ml, 4 ml to about 8 ml, 4 ml to about 9 ml, 4 ml to about 10 ml, 5 ml to about 6 ml, 5 ml to about 7 ml, 5 ml to about 8 ml, 5 ml to about 9 ml, 5 ml to about 10 ml, 6 ml to about 7 ml, 6 ml to about 8 ml, 6 ml to about 9 ml, 6 ml to about 10 ml, 7 ml to about 8 ml, 7 ml to about 9 ml, 7 ml to about 10 ml, 8 ml to about 9 ml, 8 ml to about 10 ml, or about 9 ml to about 10 ml of fixed total volume.

A medicine dropper may dispense its total volume in drops of a smaller fixed test volume. A dropper may contain about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 drops of a smaller fixed test volume. A dropper may contain at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 drops of a smaller fixed test volume. A dropper may contain no more than about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or no more than about 1 drop of a smaller fixed test volume. A dropper may contain from about 1 drop to about 2 drops, about 1 drop to about 5 drops, about 1 drop to about 10 drops, about 1 drop to about 20 drops, about 1 drop to about 30 drops, about 1 drop to about 40 drops, about 1 drop to about 50 drops, about 2 drops to about 5 drops, about 2 drops to about 10 drops, about 2 drops to about 20 drops, about 2 drops to about 30 drops, about 2 drops to about 40 drops, about 2 drops to about 50 drops, about 5 drops to about 10 drops, about 5 drops to about 20 drops, about 5 drops to about 30 drops, about 5 drops to about 40 drops, about 5 drops to about 50 drops, about 10 drops to about 20 drops, about 10 drops to about 30 drops, about 10 drops to about 40 drops, about 10 drops to about 50 drops, about 20 drops to about 30 drops, about 20 drops to about 40 drops, about 20 drops to about 50 drops, about 30 drops to about 40 drops, about 30 drops to about 50 drops, or about 40 drops to about 50 drops of a smaller fixed test volume.

The kit may further comprise a sensor device. The sensor device may be any subject sensor device as described in the present disclosure. The sensor device may be configured to measure a value of at least one biomarker in a sample. The sample may be a biological sample of an individual or may be derived from the biological sample of the individual. The biological sample may comprise: blood, plasma, serum, urine, perilymph fluid, feces, saliva, semen, amniotic fluid, cerebrospinal fluid, bile, sweat, tears, sputum, synovial fluid, vomit, bone, heart, thymus, artery, blood vessel, lung, muscle, stomach, intestine, liver, pancreas, spleen, kidney, gall bladder, thyroid gland, adrenal gland, mammary gland, ovary, prostate gland, testicle, skin, adipose, eye, brain, infected tissue, diseased tissue, malignant tissue, calcified tissue, and/or healthy tissue. The malignant tissue may comprise tumor, sarcoma, leukemia, or a derivative thereof. Alternatively or in addition to, the sensor device may be configured to collect a biological sample from the individual. In other examples, the kit may comprise one or more separate sample collector devices (e.g., a cotton swab, blood collection chip, etc.) that are configured to collect a biological sample from the individual. The one or more separate sample collector devices may be compatible with the sensor device. Alternatively, one or more separate sample collector devices may be compatible with a sensor device that is not included in the kit (e.g., as sensor device at a hospital or a pharmacy).

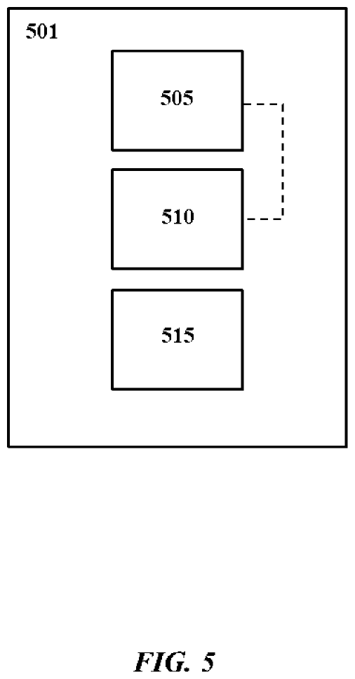

FIG. 5 illustrates an example kit 501 for determining a target dosage of a cannabinoid composition for an individual. The kit 501 may comprise a cannabinoid composition 505, an applicator device 510, and instructions for determining a target dosage of the cannabinoid composition according to any of the methods provided herein.

The cannabinoid composition 505 may be any cannabinoid composition as provided in the present disclosure. The cannabinoid composition 505 may be sufficient (e.g., may comprise sufficient volume) to determine the target dosage of the cannabinoid composition for at least 1, 2, 3, 4, 5, 6, 7, 8, 9, 10, 11, 12, 13, 14, 15, 16, 17, 18, 19, 20, 25, 30, 35, 40, 45, 50, or more times. The cannabinoid composition 505 may be sufficient to determine target dosage of the cannabinoid composition for at most 50, 45, 40, 35, 30, 25, 20, 19, 18, 17, 16, 15, 14, 13, 12, 11, 10, 9, 8, 7, 6, 5, 4, 3, 2, or 1 time. The cannabinoid composition 505 may be sufficient (e.g., may comprise sufficient volume) to determine the target dosage of the cannabinoid composition for at least 1, 2, 3, 4, 5, 6, 7, 8, 9, 10, 11, 12, 13, 14, 15, 16, 17, 18, 19, 20, 25, 30, 35, 40, 45, 50, or more individuals. The cannabinoid composition 505 may be sufficient to determine target dosage of the cannabinoid composition for at most 50, 45, 40, 35, 30, 25, 20, 19, 18, 17, 16, 15, 14, 13, 12, 11, 10, 9, 8, 7, 6, 5, 4, 3, 2, or 1 individual.

The applicator device 510 may be any applicator device as provided in the present disclosure. The applicator device 510 may be operatively coupled to the cannabinoid composition 505. In some cases, cannabinoid composition 505 may be in one or more containers, and the one or more container may be in fluid communication with the one or more containers. In some examples, the one or more containers may be a part of the applicator device 510. In alternative examples, the one or more containers may not and need not be a part of the applicator device 510. In some cases, a user may be instructed to transfer at least a portion of the cannabinoid composition 505 into the applicator device 510 to perform any of the methods as provided in the present disclosure, e.g., to determine a target dosage of the cannabinoid composition of the user.

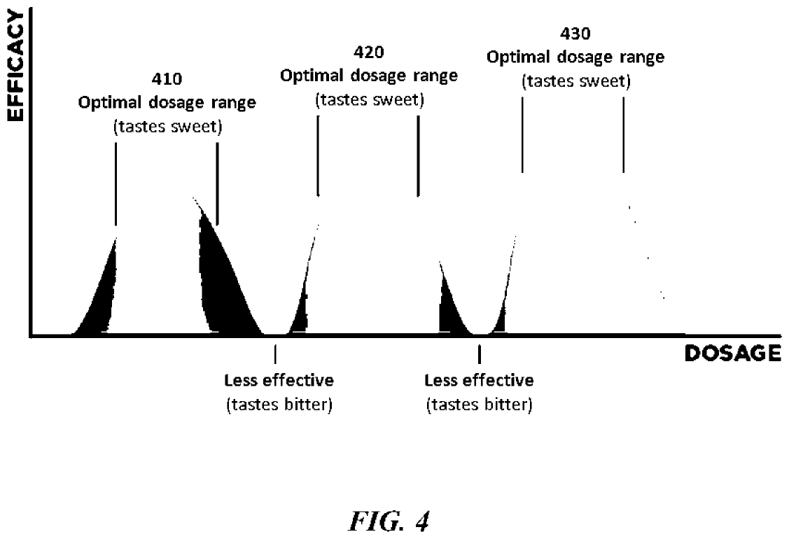

The target dosage of a cannabinoid composition may change over time. The target dosage may increase with changing health state. The target dosage may decrease with changing health state. The target dosage of a cannabinoid composition may decrease due to increased sensitivity to the effect or effects of a particular cannabinoid constituent in the composition. A cannabinoid composition may have multiple target dosages depending upon the target application (e.g. blood pressure vs. blood sugar). FIG. 4 depicts an illustration of a dosage versus efficacy curve for a microencapsulated liquid cannabinoid composition with multiple target dosages. Maximal efficacy is found at the dosage volumes corresponding to peaks 410, 420 and 430. For each optimal dosage volume, a sweet taste would be perceived by the individual, whereas the taste would become increasingly bitter once the optimal dosage has been exceeded (e.g., in a value between (i) peaks 410 and 420, (ii) peaks 420 and 430). The taste would again become sweet when a higher optimal dosage level is achieved. In some examples, multiple target dosage volumes may exist simultaneously. In other examples, multiple target dosage volumes may develop or change as the individual responds to the effects of the cannabinoid composition.

Upon determining a target dosage of a cannabinoid composition for an individual, an additional target dosage of the cannabinoid composition for the individual may be determined. The additional target dosage of the cannabinoid composition for the individual may be determined at least about 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, or more subsequent to the determination of the target dosage. The additional target dosage of the cannabinoid composition for the individual may be determined at most about 12 months, 11 months, 10 months, 9 months, 8 months, 7 months, 6 months, 5 months, 4 months, 3 months, 2 months, 4 weeks, 3 weeks, 2 weeks, 7 days, 6 days, 5 days, 4 days, 3 days, 2 days, 1 day, or less subsequent to the determination of the target dosage. At least 1, 2, 3, 4, 5, 6, 7, 8, 9, 10, 15, 20, or more additional target dosages of the cannabinoid composition may be determined for the individual. At most 20, 15, 10, 9, 8, 7, 6, 5, 4, 3, 2, or 1 additional target dosages of the cannabinoid composition may be determined for the individual.

An additional target dosage may comprise an increased amount of a cannabinoid compound as compared to a previously determined target dosage for the individual. The amount of the cannabinoid compound in the additional target dosage may be at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 100-fold, or more as compared to an amount of the cannabinoid compound in the previously determined target dosage for the individual. The amount of the cannabinoid compound in the additional target dosage may be at most about 100-fold, 50-fold, 45-fold, 40-fold, 35-fold, 30-fold, 25-fold, 20-fold, 19-fold, 18-fold, 17-fold, 16-fold, 15-fold, 14-fold, 13-fold, 12-fold, 11-fold, 10-fold, 9-fold, 8-fold, 7-fold, 6-fold, 5-fold, 4-fold, 3-fold, 2-fold, 1.9-fold, 1.8-fold, 1.7-fold, 1.6-fold, 1.5-fold, 1.4-fold, 1.3-fold, 1.2-fold, 1.1-fold, or less as compared to an amount of the cannabinoid compound in the previously determined target dosage for the individual.

An additional target dosage may comprise a decreased amount of a cannabinoid compound as compared to one or more previously determined target dosage for the individual. The amount of the cannabinoid compound in the previously determined target dosage may be at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 100-fold, or more as compared to an amount of the cannabinoid compound in the additional target dosage for the individual. The amount of the cannabinoid compound in the previously determined target dosage may be at most about 100-fold, 50-fold, 45-fold, 40-fold, 35-fold, 30-fold, 25-fold, 20-fold, 19-fold, 18-fold, 17-fold, 16-fold, 15-fold, 14-fold, 13-fold, 12-fold, 11-fold, 10-fold, 9-fold, 8-fold, 7-fold, 6-fold, 5-fold, 4-fold, 3-fold, 2-fold, 1.9-fold, 1.8-fold, 1.7-fold, 1.6-fold, 1.5-fold, 1.4-fold, 1.3-fold, 1.2-fold, 1.1-fold, or less as compared to an amount of the cannabinoid compound in the additional target dosage for the individual.

The target dosage of a cannabinoid composition may be recorded and monitored over time. Biomarkers of interest may be recorded and monitored over time in conjunction with the application of cannabinoid compositions. Target cannabinoid composition may be correlated with biomarkers of interest such as age, height, weight, resting heart rate, blood oxygen saturation, systolic blood pressure, diastolic blood pressure, basal body temperature, blood sugar, and oxidative stress level. In some aspects, correlation between one or more biomarkers and the target dosage of a particular cannabinoid composition may be used to develop a predictive model of target cannabinoid composition for a given individual. In other aspects, correlation between one or more biomarkers and the target dosage of a cannabinoid composition may be used to determine the efficacy of a cannabinoid composition for a particular application, e.g., lowering blood pressure. The target dosage of a cannabinoid composition may be correlated with about 1, 2, 3, 4, 5, 6, 7, 8, 9, or about 10 unique biomarkers. The target dosage of a cannabinoid composition may be correlated with at least about 1, 2, 3, 4, 5, 6, 7, 8, 9, or about 10 unique biomarkers. The target dosage of a cannabinoid composition may be correlated with no more than about 10, 9, 8, 7, 6, 5, 4, 3, 2, or about 1 unique biomarker.

A biomarker that is measured in association with the application of a dosage of a cannabinoid composition may be monitored and/or recorded at a particular interval. A biomarker may be measured, monitored, and/or recorded about 1, 2, 3, 4, 5, 6, 7, 8, 9, 10, 11, 12, 13, 14, 15, 16, 17, 18, 19, 20, 21, 22, 23, or about 24 times a day. A biomarker may be measured, monitored and/or recorded at least about 1, 2, 3, 4, 5, 6, 7, 8, 9, 10, 11, 12, 13, 14, 15, 16, 17, 18, 19, 20, 21, 22, 23, or about 24 times a day. A biomarker may be measured, monitored and/or recorded no more than about 24, 23, 22, 21, 20, 19, 18, 17, 16, 15, 14, 13, 12, 11, 10, 9, 8, 7, 6, 5, 4, 3, 2, or no more than about 1 time per day. A biomarker may be measured, monitored and/or recorded on a daily, weekly, or monthly interval. A biomarker may be under constant monitoring and/or recording during the application of a cannabinoid composition. A biomarker may be measured, monitored, and/or recorded on a continuous basis during the period of usage of a cannabinoid composition.

A biomarker that is measured in association with the application of a dosage of a cannabinoid composition may be monitored via a device. The device for measuring a biomarker may comprise a fixed, laboratory system such as blood analyzer. The device for measuring a biomarker may comprise a portable or handheld device such as a personal blood pressure monitor or a digital thermometer. A device for measuring a biomarker may comprise a wearable device such as a fitness watch. A measurement device for measuring a biomarker may also comprise a wireless or hardwired communication connection that permits the transfer of biomarker data to a computer or server. A measurement device for measuring a biomarker may also comprise a storage medium or a port for connecting to a storage medium to permit the export of biomarker data. Biomarkers may be measured by the individual consuming the cannabinoid composition or by a third party, such as a clinician or other medical professional.

Biomarker data and cannabinoid composition target dosage data may be transferred or uploaded into a computer system to permit monitoring, tracking, prediction, or analysis. A computer system may comprise a software system. The software system may collect data including biomarkers and consumed cannabinoid composition and dosage. The software system may perform such tasks as recording input data, analyzing data, and outputting data or prediction. A software platform may perform analyses of data to predict a target cannabinoid composition and dosage based upon biomarker data. A software platform may predict a future dosage profile for a given cannabinoid composition based upon observed changes in measured biomarkers. A software platform may predict changes in cannabinoid composition to increase the efficacy of a treatment regimen.

A software platform for monitoring or collecting target cannabinoid dosage and biomarker data may comprise a software package stored on a personal or terminal computer. A software platform for monitoring or collecting target cannabinoid dosage and biomarker data may comprise a software package stored on a remote computer system such as a server. The software platform may be web-based, such as a website or a mobile telephone app. The software platform may offer personalized tracking, monitoring, and or prediction for a single user. The software platform may collect and analyze data from a group of users to create improved correlations between measured biomarkers and the target cannabinoid dosage for a given cannabinoid composition. In some aspects, a software platform may comprise a statistical model, adaptive learning model, or a training algorithm that refines and enhances its predictive ability with increased available data.

The methods described herein may comprise a statistical analysis technique or a machine learning analysis to process data or make predictions regarding the target dosage of a cannabinoid composition and the correlated changes in measured biomarkers. Statistical methods utilized may comprise Monte Carlo methods and other types of models to provide useful information by processing input data. In some aspects, a statistical model may comprise a Hidden Markov model (HMM). An HMM may be utilized to predict a plurality of one or more hidden states from noisy observations that are dependent upon the hidden states. An HMM is defined by a set of outcomes that are state-dependent wherein the outcomes are observed and are probabilistically related to hidden states. Training an HMM produces probability distribution mapping of the hidden states to observed outcomes, allowing the state of the system to be predicted from a plurality of one or more outcomes. In order to estimate the probability of distribution of the HMM, a training algorithm may be utilized. In some examples, a training algorithm may comprise is an expectation-maximization algorithm that iteratively estimates a probability distribution until convergence with a training set's probability distribution is achieved, e.g. the Baum-Welch algorithm. In some aspects, an HMM may be used to predict the target dosage of a particular cannabinoid composition based upon a set of measured biomarkers.

Machine learning algorithms may also be utilized in order to make predictions using one or more sets of input data. In some aspects, an artificial neural network may be utilized by the model to process data or make decisions. In some aspects, an artificial neural network may comprise a feed-forward neural network, a convolutional neural network, or a recurrent neural network. Neural networks may be used to predict cannabinoid dosages by classifying data, e.g. by classifying biomarkers during cannabinoid dosing. A neural network may be trained by comparing model predictions with reference training sets. By calculating an error function, the discrepancy between model performance and reference data can be back-propagated through the neural network over several cycles to influence the value of the predicted output. A neural network may cease training when model predictions meet a convergence condition, e.g. a small error magnitude. In some aspects, multiple, layered neural networks may comprise a deep learning network, thereby increasing the predictive power of the algorithm.

Additional machine learning algorithms and statistical models may be utilized in the present disclosure. Additional machine learning algorithms may comprise logistic regressions, classification and regression tree algorithms, support vector machines, naive Bayes, K-nearest neighbors, and random forest algorithms. These algorithms may be used for many different tasks, including data classification, clustering, density estimation, or dimensionality reduction. Machine learning algorithms may be used for active learning, supervised learning, unsupervised learning, or semi-supervised learning tasks.

The present disclosure provides computer control systems that are programmed to implement methods of the disclosure. The computer control system may comprise any computer system with sufficient resources to conduct machine-learning algorithms. The computer control system may be capable of receiving and inputting data in real-time or in packets from a remote device of computer system. Data may be transmitted to the computer control system wirelessly or via a hardwired line.

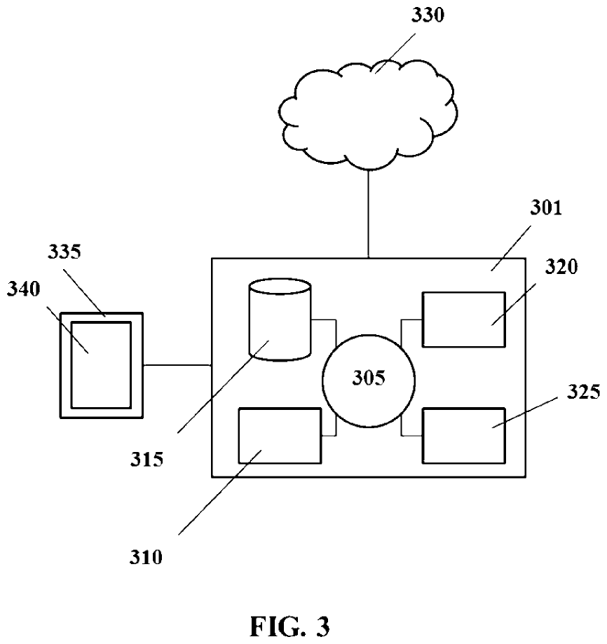

As shown in FIG. 3, the computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Example forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Methods and systems of the present disclosure can be implemented by way of one or more algorithms. An algorithm can be implemented by way of software upon execution by the central processing unit 305. The algorithm can, for example, predict a recommended daily dosage for a cannabinoid composition based upon an individual's prior dosage history and measured biomarkers.

Methods and compositions provided herein may be combined with other methods and compositions, such as those described in U.S. Pat. No. 10,350,165, U.S. Patent Publication No. 2019/0247325, and U.S. Patent Publication No. 2020/0101041, each of which is entirely incorporated herein by reference.

### EXAMPLES

### Example 1: Target Dosage Determination

An individual seeks to determine their target dosage of a given cannabinoid composition. The individual thoroughly rinses their mouth with water to clear any residual food or drink from the mouth. After rinsing the mouth, the individual prepares a full medicine dropper with the cannabinoid composition liquid. The medicine dropper has a volume of 5 ml and dispenses increments of 0.1 ml per drop. The individual applies a quarter dropper (12 to 13 drops) to the sublingual region of their mouth. The individual notes a bitter taste after application of the first quarter dropper. The individual applies a second quarter dropper and still notes a bitter taste. The individual subsequently applies a third quarter dropper of the cannabinoid composition and then notes an increasingly sweet taste, similar to honey. The individual applies more drops of the liquid to the sublingual region of the mouth in single-drop increments. After four drops, the individual no longer detects a bitter taste. The individual has determined that their target dosage of the cannabinoid composition is three quarter droppers, plus four additional drops for that particular day.

### Example 2: Target Dosage Determination with an Associated Biomarker

An individual seeks to utilize a cannabinoid composition as part of a blood pressure control regimen. The individual determines their target dosage of the cannabinoid composition according to the method described in Example 1. At the time of measuring their target cannabinoid dosage, they also measure their blood pressure using a portable pressure cuff device. Their measured blood pressure is 140/90 mmHg (systolic/diastolic). They apply the determined target dosage of the cannabinoid composition twice daily for the remainder of the week, while monitoring their blood pressure. The following week, they again perform a dosage determination using the above-described method. They measure a lowered blood pressure of 120/80 mmHg, and a correspondingly lower target cannabinoid target dosage. The individual has now determined a target dosing regimen for hypertensive and normal blood pressure states. The individual continues to administer the cannabinoid composition twice daily using a dosage based upon their blood pressure state, while occasionally recalibrating their target dosage amount using the above-described method.

### Example 3: Web-Based Dosage Determination

An individual seeks to utilize a cannabinoid composition as part of a blood pressure control regimen. The individual determines their target cannabinoid composition dosage based upon the above-described method for a chosen cannabinoid composition. The individual submits their target cannabinoid dosage measurement along with weight and blood pressure biomarker measurements to a central software server via a mobile phone app. The individual continues to measure a target dosage on a daily basis and transmit this information along with daily biomarkers to the central software system. The central software system pools their information together with information submitted by other system users. Based upon the software's training algorithm, it predicts that the individual will achieve better blood pressure control utilizing a different product and a different dosage. The individual follows the software guidance and changes composition/dosage. The individual continues to transmit dosage and biomarker data to the software, allowing the software to refine its prediction for the individual. The individual achieves stabilized blood pressure control and the software uses the individual's outcome to enhance the prediction quality for other users of the system.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-52. (canceled)

53. A method for determining a target dosage of a cannabinoid composition comprising:
(a) applying said cannabinoid composition in a first fixed volume to a region of a mouth of an individual;
(b) determining a first taste for said cannabinoid composition, wherein said first taste is characterized by one or more members selected from the group consisting of: salty, bitter, sweet, sour, and savory;
(c) applying said cannabinoid composition in a second fixed volume to said region of said mouth of said individual;
(d) determining a second taste for said cannabinoid composition, wherein said second taste is characterized by one or more members selected from the group consisting of: salty, bitter, sweet, sour, and savory; and
(e) upon detecting a change between said characterization of said second taste and said first taste, determining that a cumulative volume of said cannabinoid composition applied to said region of said individual is indicative of said target dosage of said cannabinoid composition for said individual.

54. The method of claim 53, further comprising, dispensing said cannabinoid composition in an additional fixed volume, wherein said fixed volume and said additional fixed volume are different.

55. The method of claim 53, wherein further comprising repeating (d) through (e) until said change is detected.

56. The method of claim 53, wherein said cannabinoid composition comprises cannabidiol.

57. The method of claim 53, wherein said cannabinoid composition comprises tetrahydrocannabinol at less than 0.3% by weight.

58. The method of claim 53, wherein said first fixed volume is between about 0.01 milliliters (mL) to about 5 mL.

59. The method of claim 53, wherein said second fixed volume is between about 0.01 mL to about 5 mL.

60. The method of claim 53, further comprising rinsing said mouth of said individual prior to (b) or prior to (d).

61. The method of claim 60, wherein said rinsing is prior to (b).

62. The method of claim 53, wherein said region of said mouth comprises a sublingual region or a ventral region of a tongue.

63. The method of claim 53, wherein said first taste is characterized by at least bitter.

64. The method of claim 53, wherein said second taste is characterized by at least sweet.

65. The method of claim 53, further comprising determining a second target dosage of said cannabinoid composition at a fixed interval.

66. The method of claim 53, further comprising, prior to (a), measuring a first value of at least one biomarker for said individual; and upon detecting a change between said characterization of said second taste and said first taste, measuring a second value of said at least one biomarker for said individual, wherein said determining, that a cumulative volume of said cannabinoid composition applied to said region of said individual is indicative of said target dosage of said cannabinoid composition for said individual is based at least in part on if said second value meets a condition.

67. The method of claim 66, wherein measuring said first value or said second value of at least one biomarker comprises using a sensor device to collect a biological sample of an individual and measure a value of said at least one biomarker in said biological sample.

68. The method of claim 67, wherein said biological sample comprises a blood sample.

69. The method of claim 66, wherein said at least one biomarker is selected from the group consisting of resting heart rate, blood oxygen level, systolic blood pressure, diastolic blood pressure, basal body temperature, blood sugar concentration, height, weight, body fat percentage, lean muscle mass percentage, body mass index, blood type, and resting metabolic rate.

70. The method of claim 66, wherein said condition is characterized by said second value being closer to a target value of said at least one biomarker as compared to said first value.

71. The method of claim 66, wherein said condition is characterized by said second value being further away from a target value of said at least one biomarker as compared to said first value.

72. The method of claim 66, wherein said condition is characterized by said second value being within a predetermined range around a target value of said at least one biomarker.
